(19) United States
(12) Patent Application Publication
(10) Pub. No.: US 2025/0281029 A1
(43) Pub. Date: Sep. 11, 2025

(54) PERISCOPE DEVICE FOR FORWARD VIEWING WITH A DUODENOSCOPE

(71) Applicant: Loma Linda University, Loma Linda, CA (US)

(72) Inventors: Gautam Nagendra, Loma Linda, CA (US); John F. Wetteland, Loma Linda, CA (US)

(21) Appl. No.: 18/861,148

(22) PCT Filed: Apr. 28, 2023
(86) PCT No.: PCT/US2023/066414

§ 371 Date: Oct. 28, 2024

Related U.S. Application Data
(60) Provisional application No. 63/336,740, filed on Apr. 29, 2022.

(51) Int. Cl. A61B 1/00; A61B 1/273

(52) U.S. Cl. CPC A61B 1/00179; A61B 1/273

(57) ABSTRACT

Disclosed herein is a periscope device to provide forward viewing with a duodenoscope. The periscope device includes an elongated body and a deployment mechanism. The elongated body has a proximal end and a distal end. A folding axis of the elongated body defines an elongated section and a folding section of the elongated body. The folding section is located at the distal end of the elongated body and includes a mirror attached to an outer surface. The deployment mechanism is connected to the proximal end of the elongated body and is configured to cause the folding section to rotate about the folding axis at an angle of rotation with respect to the elongated section of the elongated body.

### CROSS-REFERENCE TO RELATED APPLICATIONS

This application claims priority to U.S. Provisional Application No. 63/336,740, filed Apr. 29, 2022, the contents of which are entirely incorporated by reference herein.

### FIELD

The present disclosure relates to duodenoscopes and endoscopes for use in medical procedures. In particular, the present disclosure relates to a periscope device that temporarily allows forward viewing while using a duodenoscope.

### BACKGROUND

Duodenoscopes are specialized endoscopes that gastroenterologists use to perform endoscopic retrograde cholangiopancreatography (ERCP) procedures. Endoscopes are devices that contain a forward viewing camera at the end of a flexible tube and are used in multiple fields for minimally invasive procedures. Endoscopes contain a forward-facing camera, which provides the proceduralist with direct visualization during forward movement as the endoscope is advanced into the patient. Additionally, instruments (e.g., tools) introduced into the instrument channel of the endoscope exit the distal end of the endoscope parallel to the view of the camera, which is forward facing. Duodenoscopes, on the other hand, contain a side viewing camera, which is ideal for ERCP procedures utilized in the treatment of biliary and pancreatic diseases. Additionally, instruments introduced into the instrument channel exit the distal end of the duodenoscope perpendicular to the view of the camera. In some cases, an elevator can be adjusted to provide variability in the instrument exit angle. However, when using a duodenoscope, the proceduralist does not have direct visualization of forward movement when inserting or otherwise advancing the duodenoscope. Instead, the proceduralist must rely on the side view from the side viewing camera and the tactile feel of resistance as the duodenoscope traverses the lumen or abuts the luminal walls. Although proceduralists are extremely skilled, the lack of forward visualization while manipulating the scope during ERCP increases the chance for scope related damage or perforation injury to the patient. On the other hand, when using an endoscope, the proceduralist has direct visualization of forward movement when inserting or otherwise advancing the endoscope; however, the proceduralist does not have side visualization.

With a rising incidence of obesity in the pediatrics, there has been a greater incidence of biliary disease (such as gallstones) in the adolescent subpopulation. Similarly, there has been a rise in ERCP utilization in the pediatric population as well. Given children generally have smaller and more fragile organ systems, it is optimal to mitigate procedural risks as much as possible. The temporary conversion of the side viewing duodenoscope to provide a forward-facing view would be useful in the pediatric population where the gastrointestinal tract may be at higher risk for endoscope and manipulation related trauma.

### SUMMARY

Some aspects of the present disclosure provide a periscope device and methods of use thereof. One aspect of the present disclosure encompasses a periscope device for use with an elongate medical device, such as an endoscope or duodenoscope. The periscope device includes an elongated body and a deployment mechanism. The elongated body has a proximal end, a distal end opposite the proximal end, and an outer surface, and the elongated body defines a longitudinal axis. The elongated body can be configured to be inserted into and advanced through a working lumen of the duodenoscope. The elongated body includes a folding section that has a mirror coupled thereto. The folding section can rotate about a folding axis of the elongated body. In some aspects, deploying the deployment mechanism, which can be coupled to the proximal end of the elongated body, causes the folding section of the elongated body to rotate about a folding axis.

In some aspects, an elongated section of the elongated body is defined by the folding axis. For example, the elongated section extends from the proximal end of the elongated body to the folding axis and the folding section extends from the distal end of the elongated body to the folding axis. In some aspects, the elongated section is constructed of a flexible material and the folding section is constructed of a rigid or semi-rigid material.

In some aspects, a pin defines the folding axis and the folding section rotates about the pin. In some aspects, the folding section includes one or more knuckles and the elongated section includes one or more knuckles that correspond to the one or more knuckles of the folding section. Together, the one or more knuckles of the folding section and the one or more knuckles of the elongated section contain the pin therein, such that the folding section can rotate about the pin with respect to the elongated section. In some aspects. the elongated body has a generally circular cross section. In some aspects, the folding section and the elongated section each include one or more recesses to accommodate rotation of the folding section about the folding axis.

In some aspects, the deployment mechanism can cause the folding section to rotate about the folding axis to an angle of rotation with respect elongated section. In some examples, the angle of rotation is between 0-degrees and 180-degrees. In some aspects, the folding axis defines a fold-axis angle with respect to the longitudinal axis of the elongated body. In some examples, the fold-axis angle is approximately 45-degrees.

In some aspects, the folding section is a first folding section and the folding axis is a first folding axis. In some examples, the periscope device can include a second folding section configured to rotate about a second folding axis when the deployment mechanism is deployed. In some examples, the periscope device can include a third folding section configured to rotate about a third folding axis when the deployment mechanism is deployed. In some examples, the first folding axis, the second folding axis, and the third folding axis are each generally perpendicular to the longitudinal axis of the elongated body. In some examples. a longitudinal axis of the first folding section is generally coplanar with a longitudinal axis of the second folding section and the longitudinal axis of the second folding section is generally coplanar with a longitudinal axis of the third folding section. In some examples, the longitudinal axis of the first folding section is perpendicular to a longitudinal axis of an optical lens of the duodenoscope.

In some aspects, the elongated body defines a height after the deployment mechanism causes the folding section to rotate about the folding axis. In some examples, the height is less than 4 millimeters. In some aspects, the mirror is configured to reflect the field of view of the optical lens of a duodenoscope.

In some aspects, the deployment mechanism causes the elongated body to transition from an undeployed position to a deployed position. When the periscope device is in the undeployed position, the periscope device does not obstruct a side-facing view from the optical lens of the duodenoscope. When the periscope device is in the deployed position, the folding section reflects the side-facing view from the optical lens to provide a forward-facing view. In some examples, a longitudinal axis of the forward-facing view is generally perpendicular to a longitudinal axis of the optical lens.

In some aspects, the periscope device includes a light source coupled to the folding section of the elongated body. In some examples, the light source defines a longitudinal axis that is configured to be generally parallel to a longitudinal axis of the duodenoscope after the folding section rotates about the folding axis.

In some aspects, an angle of rotation of the folding section with respect to an elongated section of the elongated body is between 0-degrees and 180-degrees. In some aspects, the elongated body defines a length between the proximal end and the distal end. In some examples, the length is at least 124 centimeters. In some examples, the length is more than 124 centimeters. In some examples, the length is at least 124 centimeters. In some aspects, the outer surface of the elongated body has a circular cross-section that defines a diameter. In some examples, the diameter is less than 4.2 millimeters. In some aspects, the folding axis of the elongated body is oriented at approximately a 45-degree angle with respect to the longitudinal axis of the elongated body. In some aspects, the folding section of the elongated body is approximately 0.5 centimeters in length. In some aspects, the mirror is approximately 0.4 centimeters wide and 0.4 centimeters long.

In some aspects, the elongated body is constructed of one or more plastics. In some examples, the folding section of the elongated body is generally rigid and the elongated section of the elongated body is generally flexible. For example, the flexible elongated section can be curved, bent, or otherwise manipulated along the longitudinal axis.

Another aspect of the present disclosure encompasses a system for providing forward viewing with a duodenoscope. The system includes a periscope device and a duodenoscope. The periscope device is configured to be used with the duodenoscope. The duodenoscope can include an optical lens, a working lumen, and/or an elevator associated with the distal end of the working lumen. The optical lens is mounted to the outer surface near the distal end of the duodenoscope body and configured to provide imaging (e.g., side-view imaging). The distal end of the working lumen is located near the optical lens. An elevator is associated with the distal end of the working lumen and operable to adjust to an angle of elevation.

Also disclosed is a method for using the periscope device to provide forward viewing with the duodenoscope. The method includes inserting a distal end of an elongated body of the periscope device into a proximal end of a working lumen of a duodenoscope. The elongated body can include a folding axis that defines an elongated section of the elongated body and a folding section of the elongated body. The folding section of the elongated body can include a mirror coupled thereto. The method includes advancing the elongated body of the periscope device until the folding section of the elongated body extends from a distal end of the working lumen of the duodenoscope. Additionally, the method includes actuating a deployment mechanism connected to the proximal end of the elongated body of the periscope device, thereby rotating the folding section of the elongated body about the folding axis at an angle of rotation with respect to the elongated section of the elongated body. The method further includes orienting the mirror to reflect a view from an optical lens of the duodenoscope at a viewing angle.

In some aspects, the angle of rotation is between 0-degrees and 180-degrees. In some aspects, the viewing angle is approximately 45-degrees.

In some aspects, the method further includes inserting a distal end of the duodenoscope into a patient. In some aspects, the method further includes advancing the distal end of the duodenoscope to a desired location within the patient.

In some aspects, the method further includes raising an elevator of the duodenoscope to an angle of elevation to correspondingly raise at least a portion of the folding section. In some aspects, the angle of elevation is approximately 90-degrees.

In some aspects, the method further includes removing the duodenoscope from the patient. In some aspects, the method further includes removing the elongated body of the periscope device from the working lumen of the duodenoscope.

In some aspects, the method further includes inserting the distal end of the duodenoscope into a mouth of the patient. In some aspects, the method further includes advancing the duodenoscope through the mouth of the patient, through a throat of the patient, through a stomach of the patient, and into a duodenum of the patient.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that. for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIGS. 1A-1B illustrate a top view of a periscope device in an undeployed configuration and in a deployed configuration, respectively, according to one embodiment.

FIGS. 2A-2C illustrate an overview of a duodenoscope in a perspective view, a top view of the distal end of the distal end of the duodenoscope, and a perspective view of the distal end.

FIG. 3A illustrates a perspective view of the distal end of the duodenoscope after the distal end of the periscope device has been advanced through the working lumen of the duodenoscope with the elevator of the duodenoscope is in a lowered position and the periscope device in an undeployed configuration.

FIG. 3B illustrates a perspective view after the elevator has been actuated into a raised position, thereby raising the distal end of the periscope device, while the periscope device remains in an undeployed configuration.

FIG. 3C illustrates a perspective view after the periscope device has been deployed, while the elevator remains in a raised position.

FIG. 4A illustrates a side view of a periscope device in a deployed position with respect to a duodenoscope, according to one embodiment.

FIG. 4B illustrates a field of view for a duodenoscope alone, without a deployed periscope device, according to one embodiment.

FIG. 4C illustrates a field of view for a duodenoscope with a periscope device in a deployed position, according to one embodiment.

FIGS. 5A-5C illustrate a left-side view, a front view, and a right-side view. respectively, of a periscope device, according to one embodiment.

FIGS. 6A-6D illustrate perspective views of one embodiment of a periscope device being used in conjunction with a duodenoscope.

FIGS. 7A-7D illustrate perspective views of one embodiment of a periscope device being used in conjunction with a duodenoscope.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows. and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

The present disclosure generally relates to a periscope device and methods of use thereof. One aspect of the present disclosure encompasses a periscope device for use with an elongate medical device, such as an endoscope or duodenoscope. While endoscopes exist, there is a need for a periscope device that temporarily converts a side-viewing duodenoscope into a forward-viewing device, especially while the proceduralist (e.g., endoscopist) is manipulating, advancing, or otherwise positioning the duodenoscope. By providing a forward view, the periscope device can reduce the risk of mucosal injury, including perforation, to the patient by allowing the proceduralist to more safely manipulate (e.g., advance) the duodenoscope. For example, when deployed to provide a forward view, the periscope device can provide a forward-facing view that includes at least a portion of a view of the path of travel of the duodenoscope (e.g., when advancing the duodenoscope).

Provided herein is a periscope device and methods of use thereof to provide forward viewing when using a duodenoscope. The periscope device includes an elongated body and a deployment mechanism at the proximal end of the elongated body. The elongated body contains a folding axis near the distal end of the elongated body, wherein the folding axis defines an elongated section of the elongated body on the proximal side of the folding axis and a folding section of the elongated body on the distal side of the folding axis. In some embodiments, the folding section includes multiple folding axes such that the folding section includes more than one folding subsection. A mirror is attached to the outer surface of the folding section of the elongated body. When the deployment mechanism is actuated, the folding section of the elongated body rotates about the folding axis to an angle of rotation, which repositions the mirror.

The duodenoscope includes a duodenoscope body, which includes a working lumen. The distal end of the working lumen exits from the duodenoscope body near the distal end of the duodenoscope. An elevator is associated with the distal end of the working lumen, wherein the elevator is configured so that the proceduralist can raise or lower the elevator to achieve a desired angle (e.g., an angle of elevation). An optical lens is located on the outer surface (e.g., the superior surface) near the distal end of the duodenoscope and configured to provide imaging (e.g., side-view imaging).

The periscope device can be used with the duodenoscope to provide for a forward-facing view with respect to the side viewing duodenoscope. The distal end of the elongated body of the periscope device can be inserted into the proximal end of the working lumen of the duodenoscope. The elongated body can be advanced until at least the folding section of the elongated body, which contains the mirror, is beyond the distal end of the working lumen of the duodenoscope. In some embodiments, the elevator, which is associated with the distal end of the working lumen of the duodenoscope, can be raised to an angle of elevation to optimize the position (e.g., angle) of the folding section of the elongated body of the periscope device. In some aspects, the elevator can be raised until the folding section of the elongated body of the periscope device is generally perpendicular to the duodenoscope body. In other embodiments, the folding section includes more than one section (e.g., a first folding section, second folding section, and a third folding section) which each rotate about respective axes of rotation when the deployment mechanism is actuated. The deployment mechanism can be actuated, thereby causing the folding section (or multiple folding sections) of the elongated body to rotate about the respective folding axis (or axes). When the folding section rotates, the mirror on the elongated body of the periscope device repositions generally aligned with the optical lens on the outer surface of the duodenoscope. The device may be configured so that the viewing angle, the angle between the optical lens of the duodenoscope and the mirror, provides forward viewing with the duodenoscope.

The periscope device may have significant advantages over traditional duodenoscopes. As previously discussed, endoscopes are devices with a camera at the end of a flexible tube that are used in multiple fields for minimally invasive procedures. In gastroenterology, these devices can largely be divided into forward viewing endoscopes (e.g., used for standard upper endoscopies, enteroscopies, or colonoscopies) and side viewing duodenoscopes (e.g., used for cases related to and pancreatic diseases). Standard endoscopes include a forward viewing camera. Thus, when the proceduralist inserts and positions the endoscope, the proceduralist has direct visualization of where the device is going and if it is abutting another structure (e.g., the back of the throat, stomach wall, etc.). Any instrument passed through the working lumen (e.g., the port) of the endoscope exits generally parallel to the longitudinal axis of the distal end of the endoscope (e.g., the longitudinal axis of the tip of the endoscope). Standard duodenoscopes, on the other hand, include a side viewing camera. Thus, when the proceduralist inserts and positions a duodenoscope, the proceduralist does not have direct visualization of movement. Instead, the proceduralist must rely on the side view and tactile feel of resistance to know if the duodenoscope is abutting another structure (i.e., the back of the throat, stomach wall, etc.). Additionally, the duodenoscope has a dial to control an elevator that allows an instrument passed through the working lumen to exit the endoscope at different angles.

The periscope device may decrease the risk of perforation during endoscopic retrograde cholangiopacreatography (ERCP) procedures. Currently, all ERCP procedures are performed with a duodenoscope because the side viewing camera is ideal for treating certain biliary and pancreatic disorders. However, during the time it takes to advance the duodenoscope from the mouth to the duodenum, the proceduralist is manipulating the device without forward visualization, which increases the chance for endoscope related damage or perforation. Although proceduralists are skilled, there is a risk of perforation due to scope manipulation. Studies suggest that the overall perforation risk from ERCP due to scope manipulation is approximately 0.1%, whereas the general perforation rate from forward facing endoscopes is approximately 0.01%. Thus, temporarily converting the side viewing duodenoscope to a forward-facing endoscope during the positioning phase may decrease the risk of perforation in ERCPs due to duodenoscope manipulation by a factor of 10. In other words, temporarily providing forward viewing with a periscope device may allow the proceduralist to manipulate the duodenoscope safely until the ideal positioning is achieved, at which point the device can be subsequently removed and disposed. The United States Food and Drug Administration (FDA) estimates that more than 500,000 ERCPs are performed annually in the United States.

The periscope device may decrease difficulty associated with duodenal intubation. Given the peristaltic nature of the stomach, the pyloric orifice can sometimes be small or buried under gastric folds, making entry into the duodenum challenging without direct forward visualization. Thus, intubating the duodenum with the duodenoscope alone may lead to increased procedural time and risk of mucosal injury from multiple blind attempts. Temporarily allowing forward viewing utilizing the periscope device may quicken and make duodenal intubation safer by ensuring the orifice is always in view during forward advancement of the duodenoscope.

The periscope device may provide a useful training tool for learners. Most gastroenterology procedures are performed using forward facing endoscopes and colonoscopes. Thus, gastroenterology trainees are often more accustomed to the safer, forward viewing scopes. In order to expand their training to ERCPs, it is beneficial for gastroenterology trainees to experience using the side viewing duodenoscope. This transition may be made easier by augmenting the duodenoscope with forward viewing capabilities by utilizing the periscope device.

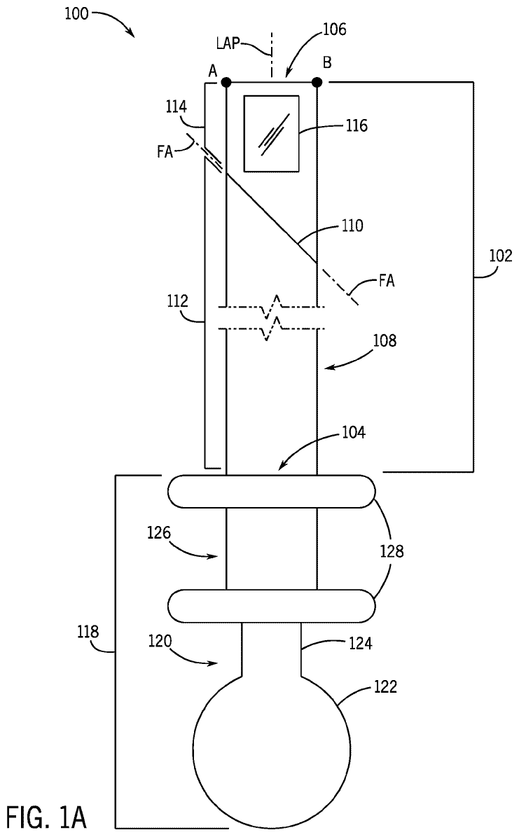

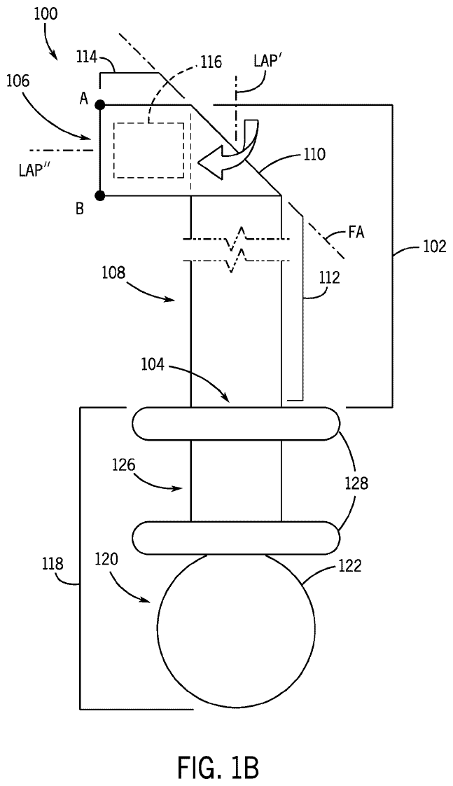

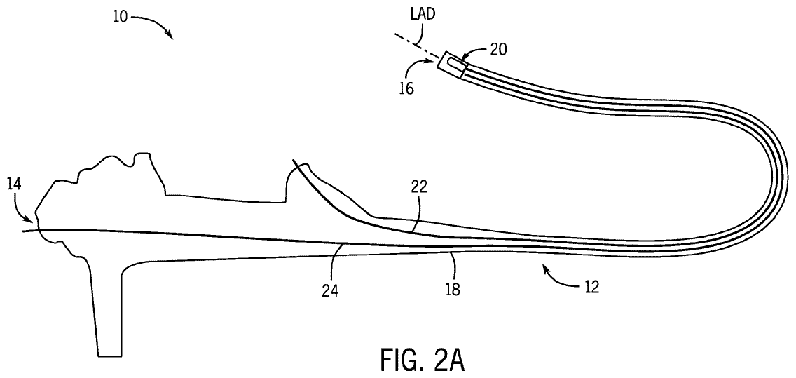

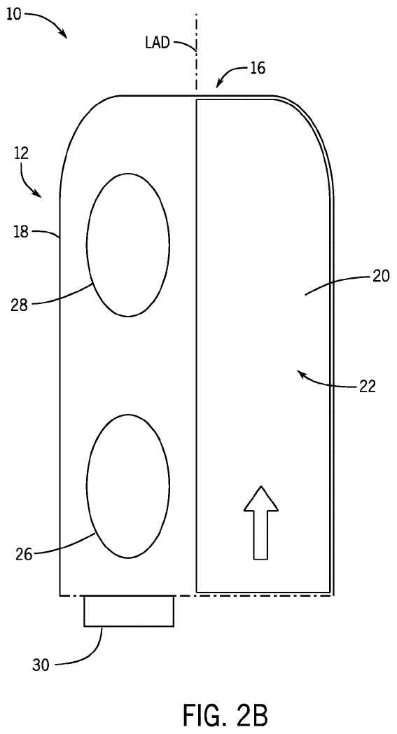

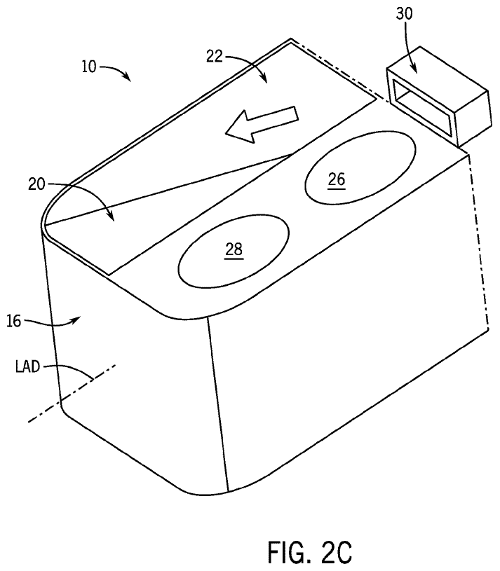

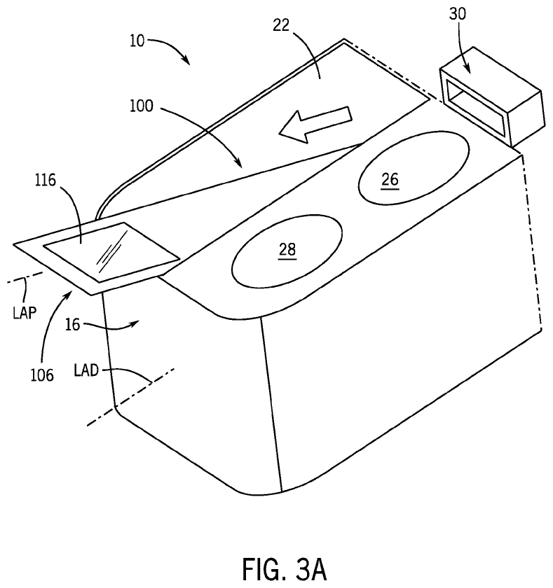

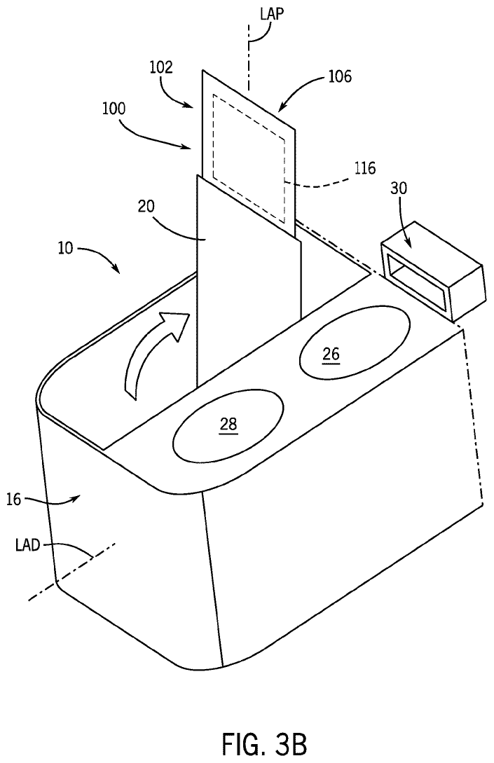

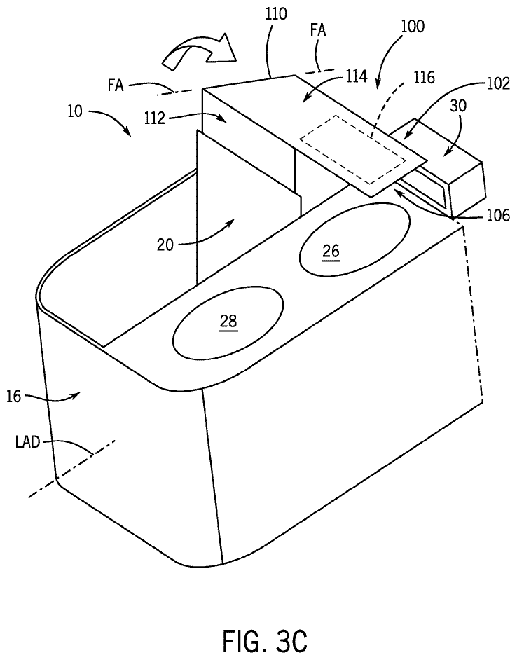

Turning to FIGS. 1A and 1B, one embodiment of the periscope device 100 is illustrated. The periscope device 100 includes an elongated body 102 and a deployment mechanism 118. The elongated body 102 and/or the deployment mechanism 118 can be made out of biocompatible materials. The elongated body 102 of the periscope device 100 is configured to be inserted into an elongate medical device (e.g., an endoscope, a duodenoscope). For example, FIGS. 2A-2C illustrate a duodenoscope 10, with FIG. 2A illustrating an overview of the duodenoscope 10 and FIGS. 2B-2C illustrating a top view and perspective view, respectively, of the distal end 16 of the duodenoscope 10. In one aspect, as illustrated for example in FIGS. 3A-3C, the periscope device 100 can be used in conjunction with a duodenoscope 10 such that the periscope device 100 can provide a forward-facing view using the side viewing duodenoscope 10.

Continuing with FIGS. 1A-1B, the elongated body 102 includes a proximal end 104, a distal end 106, and an outer surface 108. The elongated body 102 contains a longitudinal axis LAP (e.g., LAP′, LAP″). which refers to an axis along the centerline of the elongated body 102, between the proximal end 104 and the distal end 106. Although the longitudinal axis LAP of the elongated body 102 is illustrated as substantially straight in FIG. 1A, the longitudinal axis LAP of the elongated body 102 follows the path of the elongated body 102, which may be bent, curved, or otherwise manipulated. Therefore, when the elongated body 102 is manipulated during use, the longitudinal axis LAP varies with the centerline of the elongated body 102. As one example, when the elongated body 102 is inserted into and advanced through the instrument channel of a duodenoscope, the longitudinal axis LAP of the elongated body 102 can bend or curve corresponding to the longitudinal axis of the instrument channel of a duodenoscope. As another example, the longitudinal axis LAP (e.g., LAP′, LAP″) can bend or fold along a folding axis FA, as illustrated for example in FIG. 1B, such that an elongated section 112 (also referred to as a first section) defines a longitudinal axis LAP′ and a folding section 114 (also referred to as a second section) defines a longitudinal axis LAP″.

The elongated body 102 (or a portion of the elongated body 102) can be made of a flexible material such that the elongated body 102 is flexible. In this manner, the elongated body 102 of the periscope device 100 (as illustrated for example in FIGS. 1A-1B) can be inserted into and advanced through an instrument channel 22 of a duodenoscope 10 (as illustrated for example in FIGS. 2A-2C). In some embodiments, portions or sections (e.g., elongated section 112, folding section 114) of the elongated body 102 can be made out of different materials. For example, the elongated section 112 can be made out of a flexible material and the folding section 114 can be made out of a rigid or semi-rigid material. In one embodiment, the elongated body 102 (or a portion of the elongated body 102) is made of one or more polymers such as, for example, one or more natural polymers and/or one or more synthetic polymers. As non-limiting examples, the elongated body 102 can be made out of one or more plastics, silicone, rubber, latex, polyurethane elastomer, polyester elastomer, or a combination thereof. The elongated body 102 can be made out of a biocompatible material. For example, the elongated body 102 can be made out of a biocompatible plastic.

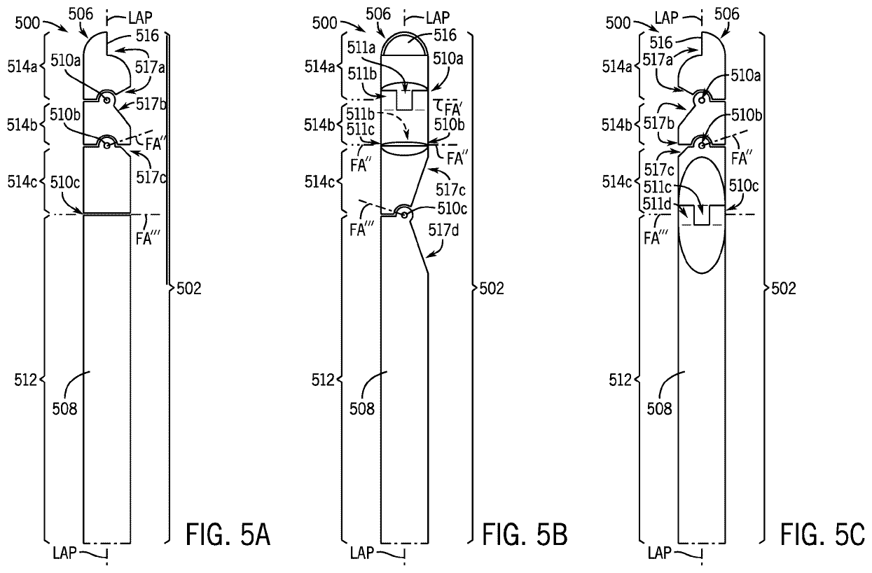

In some embodiments, portions of the elongated body 102 can be constructed out of metal (e.g., stainless steel). In some aspects, one or more pins (e.g., at the folding line 110), as illustrated for example as pins 510 (e.g., 510a, 510b, 510c) in FIGS. 5A-5C, can be constructed of metal. In some aspects, one or more internal wires or springs (e.g., along at least a portion of the longitudinal axis of the elongated body 102), as discussed below, can be constructed of metal.

The elongated body 102 defines a length, which can be measured along the longitudinal axis LA from the proximal end 104 to the distal end 106 of the elongated body 102. As one example, as illustrated for example in FIGS. 1A-1B, the length of the elongated body 102 can include both the length the elongated section 112 and the length of the folding section 114. In some embodiments, the length of the elongated body 102 is between approximately 124 centimeters and approximately 240 centimeters. In some embodiments, the length is between approximately 124 centimeters and approximately 200 centimeters. In some embodiments. the length is between approximately 124 centimeters and approximately 160 centimeters. In one embodiment, the length is approximately 124 centimeters. In one embodiment, the length is at least 124 centimeters. In one embodiment, the length is greater than 124 centimeters.

The elongated body 102 can define one or more cross sectional shapes (e.g., semi-circular, circular, ovular, triangular, rectangular, square). In some embodiments, the elongated body 102 defines one cross sectional shape. In other embodiments, the elongated body 102 defines more than one cross sectional shape. For example, portions or sections of the elongated body 102 (e.g., elongated section 112, folding section 114) can define different cross-sectional shapes. In some examples, the elongated section 112 has a generally circular cross section and the folding section 114 has a generally semi-circular cross section.

In one embodiment, the elongated body 102 contains a generally circular cross section and/or a generally semi-circular cross section such that the elongated body 102 is substantially cylindrical in shape and has a radiused outer surface 108, as illustrated for example in the elongated body 502 of the periscope device 500 in FIGS. 5A-5C as discussed below. In some aspects, the diameter of the elongated body 102 is less than or equal to the diameter of the instrument channel (also referred to as the working lumen or working channel) of the duodenoscope such that the elongated body 102 can be inserted into and advanced and/or retracted through the instrument channel. In some aspects, the diameter is between approximately 0.35 centimeters (3.5 millimeters) and 0.42 centimeters (4.2 millimeters). In some aspects, the diameter is between approximately 0.38 centimeters and 0.42 centimeters. In some aspects, the diameter is approximately 0.42 centimeters. In some aspects, the diameter is less than 0.42 centimeters.

In other embodiments, the cross section of the elongated body 102 can be ovular, triangular, rectangular, or square. For example, the elongated body can be a sheet or sheet-like such that its cross section is rectangular. In one embodiment, the elongated body 102 contains a generally square cross section that defines a width and a length that is generally the same as the width. In one embodiment, the elongated body 102 contains a generally rectangular cross-section that defines a width and a length that is less than the width. In some aspects, the width of the square or rectangular cross section of the elongated body 102 is less than or equal to the diameter of the working lumen of the duodenoscope such that the elongated body 102 may be inserted into and advanced in the working lumen. In some aspects, the width can be between approximately 0.35 centimeters and 0.42 centimeters. In some aspects. the width can be between approximately 0.38 centimeters and 0.42 centimeters. In some aspects, the width can be approximately 0.42 centimeters. In some aspects, the width can be less than 0.42 centimeters.

As illustrated for example in FIGS. 1A-1B, a folding line 110, which defines a folding axis FA, can be incorporated into the elongated body 102. The folding line 110 is generally formed across the width of the elongated body 102, and the folding line 110 can be oriented at different angles with respect to the longitudinal axis LAP of the elongated body 102 (e.g., the longitudinal axis LAP′ of the elongated section 112, the longitudinal axis LAP″ of the folding section 114). In some embodiments, the folding line 110 can define a fold-axis angle between the folding axis FA of the folding line 110 and the longitudinal axis LAP of the elongated body 102. In some aspects, the fold-axis angle can be in a range between 0-degrees (e.g., the folding axis FA is generally perpendicular to the longitudinal axis LAP) and 45-degrees. For example, the fold-axis angle can be approximately 0-degrees, 5-degrees, 10-degrees, 15-degrees, 20-degrees, 25 degrees, 30-degrees, 35-degrees, 40-degrees, or 45-degrees. FIGS. 1A-1B, for example, illustrate the fold-axis angle of the folding line 110 at approximately 45-degrees. In some aspects, the fold-axis angle can be in a range between 30-degrees and 45-degrees. In some aspects, the folding line 110 can be premanufactured into the elongated body 102. In other words, the folding line 110 is manufactured as part of the elongated body 102.

In some embodiments, the elongated body 102 includes one folding line 110, as illustrated for example in FIGS. 1A-1B. In other embodiments, the elongated body 102 includes more than one folding line 110. In some aspects, the elongated body 102 can have two folding lines 110. In some aspects, the elongated body 102 can have three folding lines 110. In some embodiments, as illustrated for example in FIGS. 5A-5C, the periscope device 500 includes more than one folding axis FA (e.g., FA′, FA″, FA′″), each of which can be defined by a pin 510 (e.g., 510a, 510b, 510c), as discussed below.

In some aspects, the folding line 110 is located near the distal end 106 of the elongated body 102 and defines an elongated section 112 of the elongated body 102 on the proximal side of the folding line 110 and a folding section 114 of the elongated body 102 on the distal side of the folding line 110. In some aspects, the folding section 114 of the elongated body 102 is located near the distal end 106 of the elongated body 102. The folding section 114 of the elongated body 102 defines a length (e.g., as measured along the longitudinal axis LAP (e.g., LAP″) of the folding section 114). In one embodiment, the length of the folding section 114 is approximately 0.5 centimeters.

When the periscope device 100 is deployed, the folding section 114 rotates about the folding line 110 (e.g., rotates about the folding axis FA) at an angle of rotation with respect to the elongated section 112 of the elongated body 102. The periscope device 100, including the elongated body 102 and folding line 110, can be configured so that the angle of rotation is an angle between 0-degrees and 180-degrees. In one embodiment, the angle of rotation is up to 180-degrees (e.g., 180-degrees or less). FIGS. 1A-1B, for example, illustrate the angle of rotation at approximately 0-degrees and 180-degrees, respectively. In one embodiment, the angle of rotation is up to 90-degrees. FIG. 3C for example, illustrates the angle of rotation at approximately 90-degrees (e.g., 90-degrees or less).

In some embodiments, the elongated body 102 or portion of the elongated body 102 (e.g., elongated section 112, folding section 114) can include a wire extending therethrough. The wire can be configured to allow the longitudinal axis LAP of the elongated body 102 to be bent, curved, or otherwise manipulated. Additionally, the wire can be configured to, when the periscope device 100 is deployed, cause the folding section 114 to rotate about the folding line 110 (e.g., the folding axis FA) with respect to the elongated section 112 of the elongated body 102. For example, the wire can be in communication with the deployment mechanism 118, such that actuating the deployment mechanism 118 causes the wire to actuate and deploy the periscope device 100. In some aspects, the wire is metal (e.g., stainless steel).

A mirror 116 is attached to the outer surface 108 of the folding section 114 of the elongated body 102. In some aspects, the mirror 116 is located near the distal end 106 of the elongated body 102. In one embodiment, the mirror 116 can be incorporated into the elongated body 102 (e.g., permanently affixed). In other embodiments, the mirror 116 can be removably attached to the elongated body 102. In one embodiment, the mirror 116 is a square shape. In other embodiments, the mirror can be a rectangular shape, a circular shape, an ovular shape, or a triangular shape. The size of the mirror 116 can vary in accordance with the size (e.g., width, diameter) of the elongated body 102. For example, the width of the mirror 116 may be equal to or less than the diameter (of an elongated body 102 having a circular cross section) or width (of an elongated body 102 having a square or rectangular cross section) of the elongated body 102. In some aspects, the mirror 116 is square and measures approximately 0.4 centimeters (i.e., 4 millimeters) in width by approximately 0.4 centimeters in length. In some aspects, the mirror 116 is square and measures less than 0.4 centimeters in width by less than 0.4 centimeters in length. In some examples, more than one mirror can be attached to the folding section 114 of the elongated body 102. In some embodiments, a rotating mechanism can be incorporated into the elongated body 102 so that the elongated body 102 (or portion of the elongated body 102) can be rotated to orient the mirror 116 in a desired position on the outer surface 108 of the elongated body 102. In other words, the rotating mechanism allows the proceduralist (also referred to as the operator) to rotate at least a portion of the elongated body 102 to adjust the axial direction that the mirror 116 is facing.

Continuing with FIGS. 1A-1B, the disclosure turns now to the deployment mechanism 118. A deployment mechanism 118 is coupled to the proximal end 104 of the elongated body 102. In one embodiment, the deployment mechanism 118 is incorporated into the periscope device 100 (e.g., permanently affixed to the elongated body 102). In other embodiments, the deployment mechanism 118 is removably coupled to the elongated body 102, such that the deployment mechanism 118 can be removed from the elongated body 102. In one embodiment, the deployment mechanism 118 includes a plunger 120 and an associated housing 126. The plunger 120 can include a rod 124 and a knob 122, with the knob 122 located at the proximal end of the rod 124. The knob 122 defines a surface, which a technician or otherwise proceduralist (e.g., endoscopist) can grip in order to actuate the plunger 120. Moreover, the surface of the knob 122 that is closest to and faces the proximal end of the housing 126 can be configured to contact the proximal end of the housing 126 when the plunger 120 is deployed (e.g., pressed inward along the longitudinal axis). The rod 124 can be configured so that deployment of the periscope device 100 (e.g., rotation of the folding section 114 about the folding axis FA with respect to the elongated section 112) occurs when the surface of the knob 122 that is closest to and faces the proximal end of the housing 126 contacts the proximal end of the housing 126. In one embodiment, the rod 124 is cylindrically shaped and the knob 122 is spherically shaped.

The housing 126 of the deployment mechanism 118 includes an interior channel that accepts the rod 124 of the plunger 120 and allows the rod 124 to actuate (i.e., move inward and outward along the longitudinal axis of the housing 126). The housing 126 can include one or more flanges 128 that extend radially outward from the housing. In one embodiment, a flange 128 is located at the proximal end of the housing 126 and a flange 128 is located at the distal end of the housing 126. The periscope device 100 is configured to deploy (e.g., the folding section 114 rotates about the folding line 110) when the operator actuates the plunger 120 (e.g., moves the rod 124 inward or outward along the longitudinal axis of the housing 126). The periscope device 100 (e.g., the elongated body 102) is configured to unfold and resume a generally linear configuration when the operator actuates the plunger 120 in the opposite direction. In one embodiment, the operator must hold the position of the plunger 120 with respect to the housing 126 to maintain a deployed configuration. For example, a spring (e.g., internal spring) can bias the plunger 120 with respect to the housing 126 so that the periscope device 100 will automatically transition from a deployed configuration (e.g., with the plunger pressed inward) to an undeployed configuration when the operator releases the plunger 120. In other embodiments, the plunger and housing may be configured so that the periscope device 100 remains in a deployed configuration until the operator manually moves the plunger 120 from a deployed position to an undeployed position, thereby causing the periscope device 100 to transition from a deployed configuration to an undeployed configuration. For example, the plunger 120 and housing 126 can contain a boss and a corresponding recessed area that allow the plunger 120 and housing to maintain a specific configuration by friction fit.

The periscope device 100 is illustrated in an undeployed configuration, for example, in FIG. 1A. The deployment mechanism 118 has not been actuated (e.g., not deployed); thus, the rod 124 of the plunger 120 is visible in the figure because the rod 124 has not been advanced into the housing 126. Because the deployment mechanism 118 has not been actuated, the folding section 114 of the elongated body 102 has not rotated about the folding line 110 (e.g., folding axis FA) with respect to the elongated section 112 of the elongated body 102. Thus, the mirror 116 is visible because FIG. 1A illustrates a top view of the periscope device 100.

The periscope device 100 is illustrated in a deployed configuration, for example, in FIG. 1B. The deployment mechanism 118 has been actuated (i.e., deployed); thus, the rod 124 of the plunger 120 is not visible in the figure because the rod 124 has been advanced into the housing 126. Because the deployment mechanism 118 has been actuated, the folding section 114 of the elongated body 102 has rotated about the folding line 110 (e.g., folding axis FA) with respect to the elongated section 112 of the elongated body 102. Thus, the mirror 116 is not visible because FIG. 1B illustrates a top view of the periscope device 100 and the folding section 114 of the elongated body 102 has been rotated approximately 180-degrees about the folding line 110 from the orientation illustrated in FIG. 1A. The translation and rotation of the distal end 106 of the elongated body 102 from an undeployed configuration to a deployed configuration is evident by the annotated point “A” and annotated point “B” illustrated on both FIGS. 1A and 1B. Annotated points “A” and “B” not physically labeled as such on the periscope device 100, rather, the points are illustrated to provide additional visualization of the rotation of the folding section 114.

The periscope device 100 can be inserted into an elongate medical device, such as a duodenoscope 10, as illustrated for example in FIGS. 2A-2C. For example, FIGS. 3A-3C illustrate the elongated body 102 of the periscope device 100 in conjunction with the duodenoscope 10. In one embodiment, the periscope device 100 (e.g., the elongated body 102) is inserted into a duodenoscope 10 (e.g., the instrument channel 22, as illustrated for example in FIG. 2A), advanced until the distal end 106 of the elongated body 102 exits the distal end of the instrument channel 22, and deployed in order to reflect the view from the optical lens 26 (also referred to as the objective lens) of the duodenoscope 10 to provide a forward-facing view. In another embodiment, the periscope device 100 can be inserted into a traditional forward-viewing endoscope and configured to provide a side-facing view from optical lens of the endoscope.

In one embodiment, as illustrated for example in FIGS. 1A-1B, the fold-axis angle (defined between the folding axis FA of the folding line 110 and the longitudinal axis LAP of the elongated body 102) of the folding line 110 is approximately 45-degrees as measured with respect to the longitudinal axis of the elongated body 102 when the elongated body 102 is substantially straight (e.g., when the longitudinal axis of the elongated body 102 is generally linear). As indicated previously, when the deployment mechanism 118 is actuated, the folding section 114 of the elongated body 102 rotates about the folding line 110 to an angle of rotation. In one example, the angle of rotation is up to approximately 180-degrees. In other words, the folding line 110 and deployment mechanism 118 are configured so that deployment of the periscope device 100 causes the folding section 114 of the elongated body 102 to rotate approximately 180-degrees about the folding line 110 with respect to the elongated section 112. Due to the fold line being approximately 45-degrees, when the folding section 114 rotates to an angle of rotation of approximately 180-degrees, the longitudinal axis LAP″ of the folding section 114 becomes oriented at approximately a 90-degree angle (e.g., generally perpendicular) with respect to the longitudinal axis LAP′ of the elongated section 112. In other words, when the folding section 114 of the elongated body 102 rotates 180-degrees about the folding line 110, the substantially straight elongated body 102 transforms from a substantially straight orientation to a substantially L-shaped orientation. In one embodiment, the mirror 116 is oriented on the outer surface 108 of the folding section 114 of the elongated body 102 so that the mirror 116 flips both horizontally and vertically when the periscope device 100 is deployed and, correspondingly, the folding section 114 of the elongated body 102 rotates approximately 180-degrees about the folding line 110 with respect to the elongated section 112. In other embodiments, the folding section 114 can be configured to rotate less than 180-degrees about the folding line 110. The degree of the angle of rotation can be adjustable, such that the angle of rotation positions the mirror 116 to provide forward viewing when the periscope device 100 is used with a duodenoscope 10, depending upon one or more of the following: the length of the folding section 114 of the elongated body 102, the angle of the elevator of the duodenoscope 10 (as discussed below), the location of the mirror 116 on the folding section 114, and the angle of the optical lens 26 relative to the longitudinal axis LAD of the duodenoscope 10.

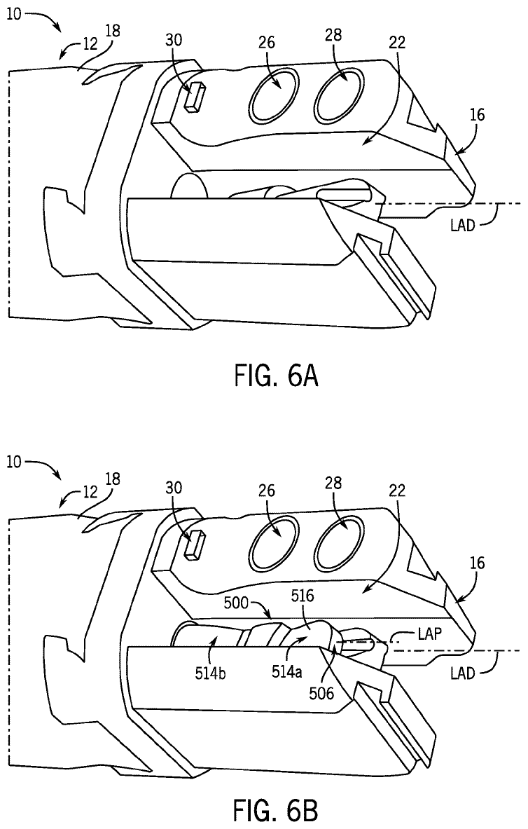

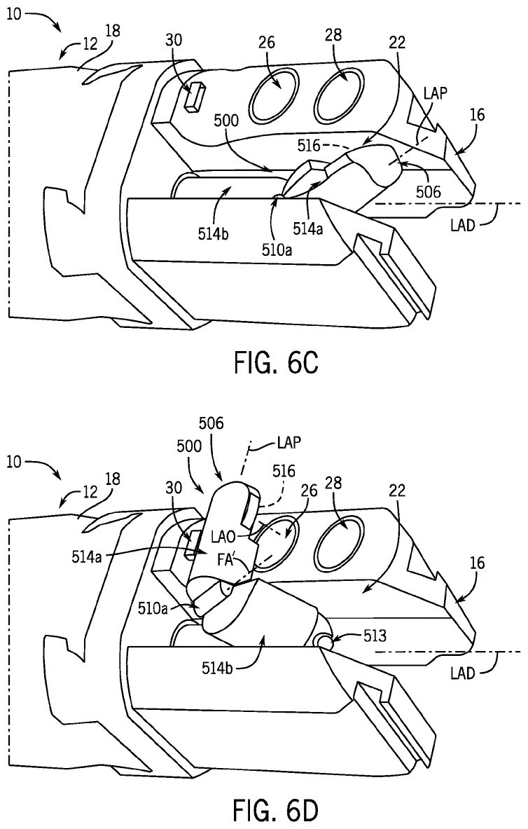

In some embodiments, the periscope device 100 includes one or more light sources, which can be configured to project light therefrom (e.g., to illuminate an area in the duodenum of the patient). In some embodiments, the light sources can be illuminated (e.g., turned on) to illuminate, or otherwise provide light to, an area such as the area that is visible when the periscope device 100 is deployed (e.g., the forward view). The one or more light sources can define a longitudinal axis. The one or more light sources can be configured so that, when illuminated, the longitudinal axis of the one or more light sources is generally parallel to the longitudinal axis of the duodenoscope (e.g., the longitudinal axis of the distal end of the duodenoscope) such that the one or more light sources can illuminate the forward-facing view provided by when the periscope device 100 is deployed. FIG. 6D, for example, illustrates one example of a light source 513 located near the second folding axis FA″ (which is not visible in FIGS. 6A-C), as discussed below. In some aspects, the light of the duodenoscope 10 (e.g., light-guide lens 28 of the duodenoscope 10, as illustrated in FIGS. 2B-2C and FIGS. 3A-3C) can be unilluminated (e.g., turned off) when the one or more light sources of the periscope device 100 are illuminated (e.g., turned on) such that the forward-facing view provided by the periscope device 100 is not blinded by the duodenoscope light.

In some embodiments, the light sources can be incorporated into or otherwise coupled to the elongated body 102 and configured to shine light outward therefrom. One or more controllers, which are in communication with the one or more light sources, can be located at or near the proximal end of the periscope device 100. The one or more controllers can control the light sources (e.g., on, off) and, in some aspects, can adjust the intensity (e.g., a dimmer) of the light sources. In other words, in some aspects, when the one or more light sources are illuminated (e.g., turned on), the intensity can be adjusted (e.g., brightened, dimmed). The light sources can receive power from an external power source (not shown in the figures). The external power source can be, as non-limiting examples, a battery, a wall outlet (e.g., in communication with the electric grid), and/or a USB port (e.g., computer USB port on an endoscopy cart).

Turning now to FIGS. 2A-2C, a duodenoscope 10 is illustrated. An overview of the duodenoscope 10 is illustrated in a perspective view in FIG. 2A. The distal end 16 of the duodenoscope 10 is illustrated in a top view and a perspective view, respectively, in FIGS. 2B-2C. The duodenoscope 10 is an elongate medical device including a body 12 that defines a longitudinal axis LAD and has a proximal end 14, a distal end 16, and an outer surface 18, as illustrated for example in the overview of duodenoscope 10 alone (e.g., without a periscope device 100) in FIG. 2A. The longitudinal axis LAD refers to an axis along the centerline of the body 12 of the duodenoscope 10, between the proximal end 14 and the distal end 16. The longitudinal axis LAD follows the path of the body 12 of the duodenoscope 10, which may be bent, curved, or otherwise manipulated. Therefore, when the body 12 of the duodenoscope 10 is manipulated during use, the longitudinal axis LAD varies with the centerline of the duodenoscope 10. When the periscope device 100 is in the instrument channel 22 of the duodenoscope 10, the longitudinal axis LAP of the periscope device 100 can bend or curve corresponding to the longitudinal axis LAD of the duodenoscope 10.

Continuing with FIG. 2A, the duodenoscope 10 can include an elevator 20 and an instrument channel 22 (which can also be referred to as a working channel or working lumen). In some aspects, the elevator 20 is positioned at the distal end 16 of the duodenoscope 10 and the elevator 20 can be actuated (e.g., raised, lowered). In some examples, an elevator wire channel 24 extends through at least a portion of the length of the body 12 of the duodenoscope 10. For example, the elevator wire can extend through the elevator wire channel 24 and be in communication with the elevator 20, such that actuating the elevator wire (e.g., advancing, retracting) causes the elevator 20 to correspondingly actuate (e.g., raise, lower). In some aspects, the instrument channel 22 extends through at least a portion of the length of the body 12 of the duodenoscope 10 and is configured to receive instruments (e.g., tools) therethrough such that instruments can be advanced or retracted within the instrument channel 22.

FIGS. 2B-2C illustrate a top view and a perspective view, respectively, of the distal end 16 of the duodenoscope 10. In some embodiments, the elevator 20 is aligned with the instrument channel 22 (e.g., the longitudinal axis of the elevator 20 is coplanar with the longitudinal axis of the instrument channel 22). In this manner, instruments (e.g., tools) that are advanced out of the distal end of the instrument channel 22 (e.g., instrument channel outlet) can come into contact with a surface of the elevator 20. Then, the position of instruments extending out of the instrument channel 22 can be adjusted via the elevator 20, as actuating the elevator 20 (e.g., raising, lowering) correspondingly raises or lowers the instrument. For example, FIGS. 2B-2C illustrate the elevator 20 in a lowered (e.g., down position).

In some embodiments, the distal end of the instrument channel 22 is adjacent to an optical lens 26 (also referred to as an objective lens). Thus, the distal end of an instrument (e.g., device, tool) is adjacent to or near the optical lens 26 after the distal end of the instrument is advanced through the distal end of the instrument channel 22 of the duodenoscope 10.

In some examples, the distal end 16 of the duodenoscope 10 can include an optical lens 26, a light-guide lens 28, and/or an air/water nozzle 30, as illustrated for example in FIGS. 2B-2C. In some aspects, the optical lens 26 is associated with a camera that provides viewing in a radially outward direction (e.g., a side view) from the distal end 16 of the duodenoscope 10. For example, the camera can provide viewing through the optical lens 26. In some aspects, the light-guide lens 28 is associated with a light source to provide lighting at the distal end 16 of the duodenoscope 10 (e.g., in a radially outward direction). In some aspects, the air/water nozzle 30 is associated with an air and/or water supply. For example, air and/or water (e.g., supplied from the corresponding air and/or water supply) can be discharged through the air/water nozzle 30. The air/water nozzle 30 can be configured such that it discharges air and/or water to clean (e.g., clear debris from, wash) the optical lens 26.

In some aspects, the distal end of the instrument channel 22 exits through the outer surface 18 of the body 12 of the duodenoscope 10 near the distal end 16 of the duodenoscope 10. In other words, the duodenoscope 10 includes an instrument channel 22 extending from a proximal end (e.g., at or near the proximal end 14 of the duodenoscope 10), along or generally parallel to the longitudinal axis LAD of the duodenoscope 10, and exiting one side of the body 12 of the duodenoscope 10 near the distal end 16 of the duodenoscope 10. The instrument channel 22 can be configured to allow the proceduralist to pass devices or tools therethrough (e.g., from the proximal end 14 of the duodenoscope 10 to the distal end 16 of the duodenoscope 10). For example, the proceduralist can insert the distal end 106 of a periscope device 100, such as periscope device 100 illustrated in FIGS. 1A-1B, into the proximal end of the instrument channel 22 of the duodenoscope 10. Then, the proceduralist can advance the periscope device 100 (e.g., the elongated body 102) until the folding section 114 (or at least a portion of the folding section 114) emerges from the distal end of the instrument channel 22.

In some aspects, the elevator 20 is associated with the distal end of the instrument channel 22. The elevator 20 is configured to be raised or lowered to provide an angle of elevation. In some embodiments, the elevator 20 controls the angle of elevation at the distal end of the instrument channel 22 (e.g., devices, instruments, or tools extending through the distal end of the instrument channel 22 correspondingly raise or lower when the proceduralist raises or lowers the elevator 20). For example, devices, instruments, or tools exiting the distal end of the instrument channel 22 can communicate or otherwise be in contact with the elevator 20. The elevator 20 directly raises or lowers the angle of the devices that are passed through the distal end of the instrument channel 22. In this manner, adjustments to the elevator 20 (e.g., changing the angle of elevation) correspondingly adjusts the angle of the device. In turn, devices can exit the distal end of the instrument channel 22 at approximately the same angle as the angle of elevation of the elevator 20. For example, the elevator 20 can be lowered, such that devices can advance from the distal end of the instrument channel 22 generally parallel to the longitudinal axis LAD of the duodenoscope 10 (e.g., the longitudinal axis of the distal end 16 of the duodenoscope 10). The elevator 20 can be raised to approximately 90-degrees, such that devices can advance from the distal end of the instrument channel 22 generally perpendicular (e.g., approximately 90-degrees) to the longitudinal axis LAD of the duodenoscope 10. In some examples, the position of the elevator 20 can be controlled by a button, lever, or dial (e.g., the button, lever, or dial is in communication with the elevator 20), which can be located at or near the proximal end 14 of the duodenoscope 10. In some embodiments, the angle of elevation, which is configured to be controlled by the proceduralist, can be adjusted to any angle between approximately 0-degrees (e.g., the angle of elevation when the elevator 20 is in the lowered position) and approximately 90-degrees (e.g., the angle of elevation when the elevator 20 in the raised position).

Various components or accessories that assist the operator in inserting, advancing, positioning, operating, or otherwise manipulating the duodenoscope 10 can be located at or near the distal end 16 of the duodenoscope 201, as previously discussed. First, an optical lens 26 can be located near the distal end 16 of the duodenoscope 10. The optical lens 26 is configured to provide imaging radially outward, away from the longitudinal axis LAD of the duodenoscope 10. For example, the optical lens 26 can be used with a camera that is configured to provide imaging. In one embodiment, the optical lens 26 is near the distal end of the instrument channel 22 and the associated elevator 20. Second, a light-guide lens 28 can be located near the distal end 16 of the duodenoscope 10. The light-guide lens 28 can be configured to provide lighting radially outward, away from the longitudinal axis LAD of the duodenoscope 10. For example, the light-guide lens 28 can be used with a light that is configured to provide lighting. The light can be configured to assist the proceduralist in obtaining imaging from the optical lens 26. In some aspects, the light-guide lens 28 is a separate component than the optical lens 26 (e.g., positioned near the optical lens 26). However, in other aspects, the light-guide lens 28 can be integrated into the optical lens 26 and/or associated camera. Third, an air/water nozzle 30 can be located near the distal end 16 of the duodenoscope 10. The air/water nozzle 30 can be configured to stream water across the optical lens 26 to clear overlying mucus or debris. For example, the air/water nozzle 30 can be in fluid communication with a water supply (which can be located at the proximal end 14 of the duodenoscope 10) and include a control (which can be controlled by the proceduralist) to control when the air/water nozzle 30 flows water onto the optical lens 26.

Turning now to FIGS. 3A-3C, a periscope device 100 (such as illustrated in FIGS. 1A-1B) is illustrated being used with a duodenoscope 10 (such as illustrated in FIGS. 2A-2C). For example, the distal end 106 of the periscope device 100 can be advanced through the instrument channel 22 of the duodenoscope 10, as illustrated for example in FIG. 3A, such that the distal end 106 of the periscope device 100 extends from the distal end of the instrument channel 22. The elevator 20 of the duodenoscope 10 can be actuated (e.g., elevated) from a lowered position to a raised position, as illustrated for example in FIG. 3B, such that a portion of the elongated body 102 of the periscope device 100 is correspondingly elevated by the elevator 20. The periscope device 100 can be deployed, as illustrated for example in FIG. 3C, such that the folding section 114 rotates about the folding line 110 (e.g., the folding axis FA) and positions the mirror 116 to reflect a view from the optical lens 26 of the duodenoscope 10 (e.g., reflect the side view to provide a forward-facing view).

The duodenoscope 10 can be inserted into and advanced in a gastrointestinal lumen (e.g., the esophagus), also referred to as the gastrointestinal tract, of a subject (e.g., a patient). During an ERCP procedure, the proceduralist (also referred to as the operator of the duodenoscope 10) inserts the distal end 16 of the duodenoscope 10 into the mouth of the patient. The operator can advance the distal end 16 of the duodenoscope 10 to a desired location within the patient. For example, the operator can advance the distal end 16 of the duodenoscope 10 so that it passes through the mouth of the patient, through the throat of the patient, and enters the stomach of the patient. The operator can further advance the distal end 16 of the duodenoscope 10 so that it passes through the stomach of the patient and enters the duodenum of the patient. The operator can use the optical lens 26 (e.g., and associated camera), the light-guide lens 28 (e.g., and associated light), the air/water nozzle 30 (e.g., and associated water supply), or a combination thereof as necessary to advance or position the distal end 16 of the duodenoscope 10 into the duodenum of the patient. For example, the operator can use the optical lens 26 and associated camera to view (e.g., side-facing view when the periscope device 100 is undeployed, forward-facing view when the periscope device 100 is deployed). As another example, the operator can use the light-guide lens 28 and associated light to provide lighting to an area being viewed. As another example, the operator can use the air/water nozzle 30 to discharge water to clean the optical lens 26, which can also clean the mirror 116 when the periscope device 100 is deployed.

In some embodiments, the periscope device 100 can be advanced, positioned, and/or deployed within the duodenoscope 10 before the duodenoscope 10 is inserted into the patient. For example, the duodenoscope 10 can be set up with the periscope device 100 advanced, positioned, and/or deployed within the duodenoscope 10 before the proceduralist (e.g., gastroenterologist, endoscopist) intubates the mouth of the patient with the duodenoscope 10.

In other embodiments, the periscope device 100 can be positioned, and/or deployed within the duodenoscope 10 after the duodenoscope 10 is inserted into the patient. For example, at any time during the advancement. positioning, or otherwise placement of the duodenoscope 10, the operator can deploy the periscope device 100. The operator inserts the distal end 106 of the elongated body 102 of the periscope device 100 into the proximal end of the instrument channel 22 of the duodenoscope 10. Then, the operator advances the elongated body 102 of the periscope device 100 until the distal end 106 of the elongated body 102 exits the distal end of instrument channel 22 of the duodenoscope 10, as illustrated for example in FIG. 3A. The elevator 20 can be in a lowered position. In one embodiment, the operator advances the elongated body 102 until the folding section 114 of the elongated body, which contains the mirror 116, exits the distal end of instrument channel 22 of the duodenoscope 10. The mirror 116 can be configured so that the mirror 116 is facing radially outward, away from the longitudinal axis LAD of the duodenoscope 10 when the distal end 106 of the elongated body 102 of the periscope device 100 exits the distal end of instrument channel 22 of the duodenoscope 10. Therefore, after the folding section 114 of the elongated body 102 is elevated with the elevator 20 and the folding section 114 is rotated about the folding line 110 (e.g., when the periscope device is deployed) the mirror 116 is generally facing the optical lens 26 of the duodenoscope 10. In some embodiments, such as illustrated in FIGS. 6A-6D, the folding section 114 (or a portion of the folding section 114) can be elevated without an elevator 20. For example, as illustrated in FIGS. 5A-5C, the periscope device 500 can include more than one folding axis FA, such as a first folding axis FA′, a second folding axis FA″, and a third folding axis FA′″. Rotation about one or more folding axis FA (or axes) can properly position the periscope device 500 without the use of an elevator 20 in the duodenoscope 10.

The operator can raise the elevator 20, as illustrated for example in FIG. 3B, such as by manipulating a button, lever, or dial (which can be located at or near the proximal end 14 of the duodenoscope 10) that is in communication with the elevator 20, to provide an angle of elevation of the elevator 20. When the operator raises the elevator 20, correspondingly, the distal end 106 of the elongated body 102 of the periscope device 100 raises (e.g., the elevator 20 raises the distal end 106 of the elongated body 102). As the distal end 106 raises, the longitudinal axis LAP (e.g., LAP″) at the distal end 106 of the periscope device 100 becomes less parallel and more perpendicular to the longitudinal axis LAD of the duodenoscope 10. In one embodiment, the elevator 20 is raised until the longitudinal axis LAP at the distal end 106 of the elongated body 102 of the periscope device 100 is approximately 90-degrees (e.g., generally perpendicular) from the longitudinal axis LAD of the duodenoscope 10 (e.g., the longitudinal axis at the distal end 16 of the duodenoscope 10). For example, the longitudinal axis LAP″ of the folding section 114 of the elongated body 102 can be generally perpendicular to the longitudinal axis LAD of the duodenoscope 10. In other embodiments, the angle of elevation of the elevator 20 can be less than 90-degrees. For example, the angle of the elevation of the elevator 20 can be adjusted, to provide forward viewing with the duodenoscope 10, depending on one or more of the following: the length of the folding section 114 of the elongated body 102 of the periscope device 100, the location of the mirror 116 on the folding section 114, and the degree to which the folding section 114 rotates about the elongated section 112 of the elongated body 102.

The operator can deploy the periscope device 100, as illustrated for example in FIG. 3C. In other words, the operator actuates the deployment mechanism 118 (e.g., by pressing the plunger 120 into the housing 126 of the deployment mechanism 118), as illustrated in FIGS. 1A-1B. This causes the folding section 114 of the elongated body 102 of the periscope device 100 to rotate about the folding line 110 and orients the mirror 116 of the periscope device 100 so that it generally faces back towards the optical lens 26 of the duodenoscope 10, as illustrated for example in FIG. 3C.

A viewing angle is defined by an angle measured between a plane of the optical lens 26 (e.g., the plane is perpendicular to the longitudinal axis of the optical lens 26) and a plane of the mirror 116 (e.g., planar surface of the mirror) with respect to the forward viewing direction. In one embodiment, the viewing angle is can range between approximately 25-degrees and 75-degrees. In one embodiment, the viewing angle is approximately 45-degrees (e.g., the surface of the mirror 116 faces forward toward the distal end 16 of the duodenoscope 10 at an approximately 45-degree angle with respect to the optical lens 26). In other words, the length of the folding section 114 of the elongated body 102 of the periscope device 100, the degree that the folding section 114 rotates about the folding line 110, the angle of the elevator 20 of the duodenoscope 10, and the location of the mirror 116 on the folding section 114 are configured so that the viewing angle (e.g., the angle of the mirror 316 with respect to the optical lens 307) provides a forward-facing view when the periscope device 100 is deployed.

When the periscope device 100 is deployed, the mirror 116 provides forward viewing for the duodenoscope 10. This provides the operator with a forward view, similar to the viewing that would be provided by a traditional forward-viewing endoscope, when using the traditional side-viewing duodenoscope 10. In effect, the forward viewing enhances the ability of the operator to advance, manipulate, or otherwise operate the duodenoscope 10. In one embodiment, the periscope device 100 is deployed only temporarily, thereby temporarily converting the duodenoscope 10 into a forward-viewing device. For example, once the distal end 16 of the duodenoscope 10 is positioned at a desired location within the body of the patient, the operator can un-deploy (i.e., deactivate) the periscope device 100. In some cases, the operator can remove the periscope device 100 from the duodenoscope 10 (e.g., retract the elongated body 102 of the periscope device 100 through the instrument channel 22 of the duodenoscope 10).

FIG. 3A illustrates the periscope device 100 in an undeployed position and the elevator 20 in a lowered position when the distal end 106 of the elongated body 102 of the periscope device 100 exits the distal end of the instrument channel 22 of the duodenoscope 10. Because the deployment mechanism has not been actuated, the folding section 114 of the elongated body 102 of the periscope device 100 has not rotated about the folding line 110 with respect to the elongated section 112 of the elongated body 302. Thus, the mirror 116 is visible because FIG. 3A illustrates a perspective view, in which a portion of the top view is visible, of the distal end 106 of the periscope device 100 advanced to the distal end 16 of the duodenoscope 10.

FIG. 3B illustrates the elevator 20 in a raised position and, correspondingly, the distal end 106 of the elongated body 102 of the periscope device 100 in a raised position. The longitudinal axis LAP of the distal end 106 of the elongated body 102 of the periscope device 100 is oriented generally perpendicular to the longitudinal axis LAD of the distal end 16 of the duodenoscope 10. The mirror 116 is not visible (so it is illustrated with dashed lines); however, the backside of the folding section 114 of the elongated body 102 of the periscope device 100 is visible, due to the orientation of the perspective view.

FIG. 3C illustrates the periscope device 100 in a deployed position. The elevator 20 is in a raised position; therefore, deploying the periscope device 100 causes the folding section 114 of the elongated body 102 of the periscope device 100 to rotate about the folding line 110 (e.g., folding axis FA). In other words, the folding section 114 of the elongated body 102 of the periscope device 100 transitions to form an L-shape with respect to the elongated section 112 of the elongated body 102. After the folding section 114 has rotated with respect to the elongated section 112, the face of the mirror 116 is generally facing towards the optical lens 26.

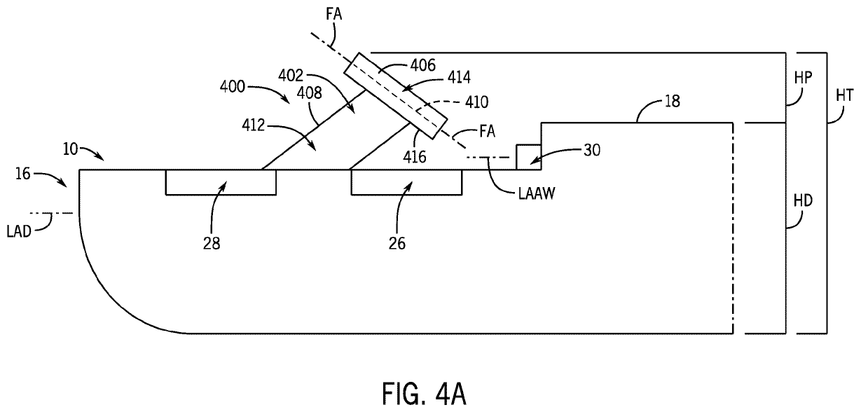

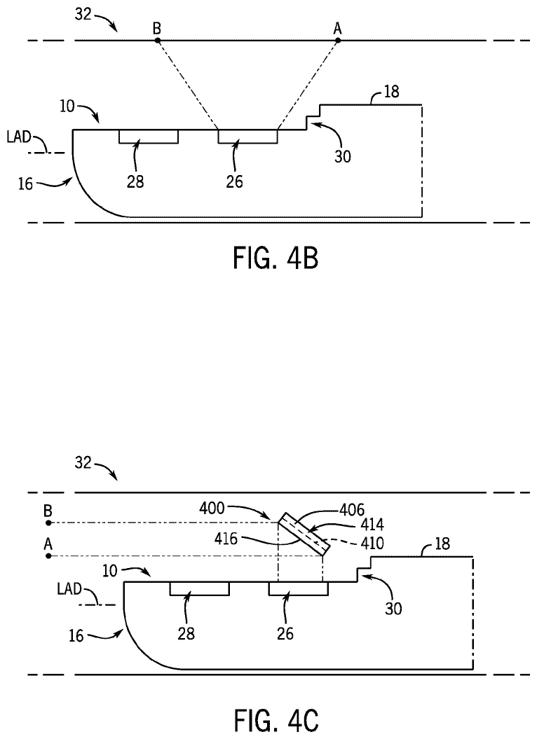

Turning now to FIGS. 4A-4C, side views of one embodiment of a periscope device 400 are illustrated. The periscope device 400 is illustrated in a deployed position with respect to a duodenoscope 10 in the side view illustrated in FIG. 4A. The field of view for a duodenoscope 10 alone (e.g., without a periscope device 400) and the field of view with the periscope device 400 in the deployed position are illustrated in the side views of FIGS. 4B-4C, respectively. The periscope device 400 illustrated in FIGS. 4A-4C can include one or more same or similar features as the periscope device 100 illustrated in FIGS. 1A-3C. Due to the same or similar features, the reference numbers and corresponding descriptions provided above for various components, elements, portions, etc., included in the periscope device 100 in FIGS. 1A-3C can be generally applied to the same or similar components, elements, portions, etc., included in the periscope device described in FIGS. 4A-4C; however, the reference numbers in FIGS. 4A-4C are 400 series rather than 100 series.

The periscope device 400 (e.g., elongated body 402) illustrated in FIGS. 4A-4C can be advanced through the working channel of a duodenoscope 10. Then, the periscope device 400 can be deployed (as illustrated in FIG. 4A and FIG. 4C) such that the mirror 416 reflects the field of view from the optical lens 26 of the duodenoscope 10. In some aspects, deploying the periscope device 400 causes the second section 414 of the elongated body 402 of the periscope device 400 to rotate about a folding axis FA, which can be defined by a pin 410. Because FIG. 4A illustrates the periscope device 400 in a deployed position, the distal end 406 of the second section 414 and the outer surface 408 of the first section 412 are visible in the figure. In some aspects, in the deployed position, the longitudinal axis of the second section 414 is generally perpendicular to the longitudinal axis of the first section 412. When deployed, the periscope device 400 can provide a forward-facing view (e.g., reflected view) with the optical lens 26 of a side-viewing duodenoscope 10. In FIG. 4A and FIG. 4C, the second section 414 of the elongated body 402 of the periscope device 400 is illustrated with a generally rectangular cross section. As previously, discussed, the cross-sectional shape of the elongated body 402 (e.g., first section 412, second section 414) can be various shapes (e.g., circular, rectangular). In some examples, the first section 412 of the elongated body 402 can have a generally circular cross section and the second section 414 can have a generally rectangular cross section.

FIG. 4A illustrates a side view of the periscope device 400 in a deployed position with respect to the duodenoscope 10. The duodenoscope 10, by itself, defines define a height HD that is measured from a bottom-side outer surface 18 of the duodenoscope 10 to a top-side outer surface 18. In some aspects, the height HD can be referred to as the diameter of the duodenoscope 10. When the periscope device 100 is deployed, as illustrated for example in FIG. 4A, the periscope device 100 can define a height HP that is measured from the top-side outer surface 18 of the duodenoscope 10 to the most radially-outward point (e.g., highest point) on the periscope device 400. The height HD of the duodenoscope 10 and the height HP of the periscope device 400, together, define a total height HT.

In some aspects, the periscope device 400 is configured such that the total height HT is less than the diameter of a gastrointestinal lumen (e.g., esophagus). In some aspects, when the periscope device 400 is deployed, the height HP of the periscope device 400 (e.g., the elongated body 402) is less than 4 millimeters (mm). In some aspects, the height HP is less than 2 mm. In some aspects, the height HP of the periscope device 400 is configured such that discharging air and/or water from the air/water nozzle 30 cleans (e.g., clears debris from, washes) the mirror 416. As previously discussed, the air/water nozzle 30 can discharge air and/or water to clean the optical lens 26 of the duodenoscope 10. Thus, in some aspects, discharging air and/or water from the air/water nozzle 30 cleans both the mirror 416 of the periscope device 400 and the optical lens 26 of the duodenoscope 10. For example, the discharged air and/or water can define a longitudinal axis LAAW of the air/water nozzle 30. The height HP of the periscope device 400 can be configured so that discharging water from the air/water nozzle 30 causes the discharged water to flow over both the surface of the mirror 416 and the surface of the optical lens 26 due to the proximity and/or position of the mirror 416 with respect to the optical lens 26.

FIGS. 4B-4C illustrate a representative field of view for both the duodenoscope 10 alone (e.g., with the periscope device 400 in an undeployed position) and the duodenoscope 10 in conjunction with the periscope device 400 (e.g., with the periscope device 400 in a deployed position), respectively. The field of view in FIGS. 4B-4C is illustrated by the dashed lines extending from the outer edges of the optical lens 26 of the duodenoscope 10 to the points “A” and “B.” Additionally, in FIGS. 4B-4C, the distal end 16 of the duodenoscope 10 illustrated within a gastrointestinal lumen 32 (e.g., the esophagus). As previously discussed, with the periscope device 400 in an undeployed position, the duodenoscope 10 can be advanced to a desired location within a gastrointestinal lumen 32. Then, after the duodenoscope 10 reaches the desired location, the periscope device 400 can be deployed.

With the periscope device 400 in an undeployed position, as illustrated for example in FIG. 4B, the field of view (represented by the dashed lines extending from the optical lens 26 to the points “A” and “B”) from the optical lens 26 of the duodenoscope 10 is a side-facing view. In this manner, the optical lens 26 (e.g., the associated camera) provides a view of an inner wall of the gastrointestinal lumen 32. In some aspects, with the periscope device 400 in an undeployed position, a longitudinal axis of the field of view is generally perpendicular to the longitudinal axis LAD of the duodenoscope 10. The longitudinal axis of the field of view can be coincident with the longitudinal axis of the optical lens 26. In some aspects, with the periscope device 400 in an undeployed position, the longitudinal axis of the field of view is generally perpendicular to the inner wall of the gastrointestinal lumen 32.

With the periscope device 400 in a deployed position, as illustrated for example in FIG. 4C, the field of view (represented by the dashed lines extending from the optical lens 26 to the points “A” and “B”) of the optical lens 26 of the duodenoscope 10 and reflected by the mirror 416 of the periscope device 400 is a forward-facing view. In this manner, the mirror 416 of the periscope device 400 provides a reflected view (e.g., a forward view) from the optical lens 26 (e.g., the associated camera) of the duodenoscope 10. It should be noted that the first section 412 (as illustrated in FIG. 4A) is not illustrated in FIG. 4C in order to clearly illustrate the field of view; however, the second section 414 is supported by a first section 412. It should also be noted that the points “A” and “B” are representative of the forward or reflected view to a distance. In some aspects, with the periscope device 400 in a deployed position, a longitudinal axis of the field of view is generally perpendicular to the longitudinal axis of the optical lens 26. In some aspects, with the periscope device 400 in a deployed position, the longitudinal axis of the field of view is generally parallel to the longitudinal axis LAD of the duodenoscope 10. In some aspects, with the periscope device 400 in a deployed position, a longitudinal axis of the field of view is generally parallel to the inner wall of the gastrointestinal lumen 32.

In some embodiments, a display monitor can display the view provided by the optical lens 26 (e.g., via the associated camera) of the duodenoscope 10. In other words, the field of view through the optical lens 26 can be displayed on a monitor. For example, the points “A” and “B” as illustrated in FIG. 4B with the periscope device 400 not deployed and FIG. 4C with the periscope device 400 deployed can be displayed on the monitor. In one example, the monitor can display point “A” as the top point of the view on the display and point “B” as the bottom point of the view on the display. In some aspects, the monitor can display a view (e.g., monitor view) that is inverted from a vertical axis. For example, the monitor view can be inverted with respect to the field of view.

Turning now to FIGS. 5A-5C, one embodiment of the periscope device 500 is illustrated. The periscope device 500 is illustrated in a left-side view, a front view, and a right-side view, respectively, in FIGS. 5A-5C. The periscope device 500 illustrated in FIGS. 5A-5C can include one or more same or similar features as the periscope device 100 illustrated in FIGS. 1A-3C and/or the periscope device 400 illustrated in FIGS. 4A-4C. Due to the same or similar features, the reference numbers and corresponding descriptions provided above for various components, elements, portions, etc., included in the periscope device 100 in FIGS. 1A-3C and the periscope device 400 in FIGS. 4A-4C can be generally applied to the same or similar components, elements, portions, etc., included in the periscope device described in FIGS. 5A-5C; however, the reference numbers in FIGS. 5A-5C are 500 series rather than 100 series or 400 series.

The periscope device 500 includes an elongated body 502 that extends from a proximal end to a distal end 506 and has an outer surface 508. The elongated body 502 defines a longitudinal axis LAP. In some aspects, the proximal end of the elongated body 502 includes a deployment mechanism such as, for example, the deployment mechanism 118 illustrated in FIGS. 1A-1B. The deployment mechanism can be configured to cause the elongated body 502 to actuate (e.g., transition from an undeployed position to a deployed position) when the deployment mechanism is actuated.

The elongated body 502 of the periscope device 500 includes an elongated section 512 and a folding section 514 (e.g., 514a, 514b, 514c). In some aspects, the elongated body 502 (e.g., the elongated section 512, the folding section 514) defines a generally circular cross section. In some aspects, the elongated section 512 is constructed of a flexible material, such that the longitudinal axis of the elongated section 512 can be bent, curved, or otherwise manipulated. In some aspects, the folding section 514 is constructed of a rigid or semi-rigid material, such that the longitudinal axis of the folding section 514 remains generally linear even when the longitudinal axis of the folding section 514 is rotated as discussed below.

The folding section 514 of the elongated body 502 of the periscope device 500, as illustrated in FIGS. 5A-5C, includes three folding sections 514 (e.g., 514a, 514b, 514c), which can be referred to as a first folding section 514a, a second folding section 514b, and a third folding section 514c. The periscope device 500 includes three folding axes FA (e.g., FA′, FA″, FA′″), which can be referred to as a first folding axis FA′, a second folding axis FA″, and a third folding axis FA′″. Each of the folding sections 514 can rotate about the corresponding folding axis FA. In some aspects, each folding axis FA is defined by a pin 510 (e.g., 510a, 510b, 510c). For example, each pin 510 can be a cylindrical body (e.g., a rod) that secures the elongated body 502 (e.g., the respective folding sections 514) together, while also allowing rotation of the folding section 514 of the elongated body 502 about the pin 510. In some aspects. each pin 510 is positioned within knuckles 511 (e.g., 511a, 511b, 511c) of the adjacent folding sections 514. In some aspects, each pin 510 is constructed of metal, such as, for example, stainless steel.

Continuing with FIGS. 5A-5C, the disclosure now turns to the first folding section 514a. The first folding section 514a can include a mirror 516, which is configured to provide a reflected view when the periscope device 500 is deployed to position the mirror over the optical lens of a duodenoscope. The first folding section 514a is rotatably coupled to the second folding section 514b along a first folding axis FA′. In this manner, the first folding section 514a can rotate about the first folding axis FA′ with respect to the second folding section 514b. In some aspects, the first folding axis FA′ is generally perpendicular to the longitudinal axis LAP of the periscope device 500 (e.g., the longitudinal axis of the folding section 514). For example, the first folding axis FA′ can be generally perpendicular to the longitudinal axis of the first folding section 514a and/or generally perpendicular to the longitudinal axis of the second folding section 514b. As previously discussed, the first folding section 514a can be constructed of a rigid or semi-rigid material, such that the longitudinal axis of the first folding section 514a remains generally linear even when the first folding section 514a is rotated about the first folding axis FA′.

In some aspects, the first folding axis FA′ is defined by a first pin 510a. In other words, the first pin 510a secures the first folding section 514a to the second folding section 514b, while allowing the first folding section 514a to rotate about the first folding axis FA′. In some aspects, as best illustrated in FIG. 5B, the proximal end of the first folding section 514a can include one or more knuckles 511a and the distal end of the second folding section 514b can include one or more knuckles 511b. The first pin 510a can join the knuckles 511a, 511b (e.g., the knuckles 511a, 511b can receive the first pin 510a therethrough) to rotatably couple the first folding section 514a to the second folding section 514b.

A first angle of rotation can be defined as the angle between the longitudinal axis of the first folding section 514a and the longitudinal axis of the second folding section 514b. In other words, the first angle of rotation changes as the first folding section 514a rotates about the first folding axis FA′ with respect to the second folding section 514b. In some aspects, the first angle of rotation can range between 180-degrees (i.e., the longitudinal axis of the first folding section 514a is generally parallel to or colinear with the longitudinal axis of the second folding section 514b) to approximately 90-degrees (i.e., the longitudinal axis of the first folding section 514a is generally perpendicular to the longitudinal axis of the second folding section 514b). In some aspects, when the periscope device 500 is in an undeployed position, the first angle of rotation is generally 180-degrees. In some aspects, when the periscope device 500 is in a deployed position, the first angle of rotation is between 60-degrees and 120-degrees. In some aspects, when the periscope device 500 is in a deployed position, the first angle of rotation is generally 90-degrees.

In some aspects, the first folding axis FA′ is generally perpendicular to both the longitudinal axis of the first folding section 514a and the longitudinal axis of the second folding section 514b, regardless of the first angle of rotation. For example, when the first angle of rotation is generally 180-degrees (e.g., when the periscope device 500 is undeployed), the first folding axis FA′ is generally perpendicular to both the longitudinal axis of the first folding section 514a and the longitudinal axis of the second folding section 514b. When the first angle of rotation is generally 90-degrees (e.g., when the periscope device 500 is deployed), the first folding axis FA′ is generally perpendicular to both the longitudinal axis of the first folding section 514a and the longitudinal axis of the second folding section 514b. In some aspects, the longitudinal axis of the first folding section 514a is generally coplanar with the longitudinal axis of the second folding section 514b, regardless of the first angle of rotation. For example, when the first angle of rotation is generally 180-degrees, the longitudinal axis of the first folding section 514a is generally coplanar with the longitudinal axis of the second folding section 514b. When the first angle of rotation is generally 90-degrees, the longitudinal axis of the first folding section 514a is generally coplanar with the longitudinal axis of the second folding section 514b.

The first folding section 514a can include one or more recesses 517 (e.g., 517a), as best illustrated in FIG. 5A and FIG. 5C. Each of the recesses 517a define a portion of the first folding section 514a that has a cross-sectional area that is less than the cross-sectional area of the elongated body 502. In some aspects, the distal end of the first folding section 514a can include a recess 517a that defines a planar surface, although the outer surface 508 of the elongated body 502 rounded such that it defines a radius. The mirror 516 (e.g., generally planar mirror 516) can be positioned on the planar surface. In some aspects, the proximal end of the first folding section 514a includes a recess 517a such that the first folding section 514a can rotate with respect to the second folding section 514b.

Continuing with FIGS. 5A-5C, the disclosure now turns to the second folding section 514b. The second folding section 514b is rotatably coupled to the third folding section 514c along a second folding axis FA″. In this manner, the second folding section 514b can rotate about the second folding axis FA″ with respect to the third folding section 514c. In some aspects, the second folding axis FA″ is generally perpendicular to the longitudinal axis LAP of the periscope device 500 (e.g., the longitudinal axis of the folding section 514). For example, the second folding axis FA″ can be generally perpendicular to the longitudinal axis of the second folding section 514b and/or generally perpendicular to the longitudinal axis of the third folding section 514c. In some aspects, the second folding axis FA″ is generally parallel to the first folding axis FA′. As previously discussed, the second folding section 514b can be constructed of a rigid or semi-rigid material, such that the longitudinal axis of the second folding section 514b remains generally linear even when second folding section 514b is rotated about the second folding axis FA″.

In some aspects, the second folding axis FA″ is defined by a second pin 510b. In other words, the second pin 510b secures the second folding section 514b to the third folding section 514c, while allowing the second folding section 514b to rotate about the second folding axis FA″. In some aspects, as best illustrated in FIG. 5B, the proximal end of the second folding section 514b can include one or more knuckles 511b and the distal end of the third folding section 514c can include one or more knuckles 511c. The second pin 510b can join the knuckles 511b. 511c (e.g., the knuckles 511b, 511c can receive the second pin 510b therethrough) to rotatably couple the second folding section 514b to the third folding section 514c.

A second angle of rotation can be defined as the angle between the longitudinal axis of the second folding section 514b and the longitudinal axis of the third folding section 514c. In other words, the second angle of rotation changes as the second folding section 514b rotates about the second folding axis FA″ with respect to the third folding section 514c. In some aspects, the second angle of rotation can range between 180-degrees (i.e., the longitudinal axis of the second folding section 514b is generally parallel to or colinear with the longitudinal axis of the third folding section 514c) to approximately 90-degrees (i.e., the longitudinal axis of the second folding section 514b is generally perpendicular to the longitudinal axis of the third folding section 514c). In some aspects, when the periscope device 500 is in an undeployed position, the second angle of rotation is generally 180-degrees. In some aspects, when the periscope device 500 is in a deployed position, the second angle of rotation is between 60-degrees and 120-degrees. In some aspects, when the periscope device 500 is in a deployed position, the second angle of rotation is generally 90-degrees.

In some aspects, the second folding axis FA″ is generally perpendicular to both the longitudinal axis of the second folding section 514b and the longitudinal axis of the third folding section 514c, regardless of the second angle of rotation. For example, when the second angle of rotation is generally 180-degrees (e.g., when the periscope device 500 is undeployed), the second folding axis FA″ is generally perpendicular to both the longitudinal axis of the second folding section 514b and the longitudinal axis of the third folding section 514c. When the second angle of rotation is generally 90-degrees (e.g., when the periscope device 500 is deployed), the second folding axis FA″ is generally perpendicular to both the longitudinal axis of the second folding section 514b and the longitudinal axis of the third folding section 514c. In some aspects, the longitudinal axis of the second folding section 514b is generally coplanar with the longitudinal axis of the third folding section 514c, regardless of the second angle of rotation. For example, when the second angle of rotation is generally 180-degrees, the longitudinal axis of the second folding section 514b is generally coplanar with the longitudinal axis of the third folding section 514c. When the second angle of rotation is generally 90-degrees, the longitudinal axis of the second folding section 514b is generally coplanar with the longitudinal axis of the third folding section 514c.

The second folding section 514b can include one or more recesses 517 (e.g., 517b), as best illustrated in FIG. 5A and FIG. 5C. Each of the recesses 517b define a portion of the second folding section 514b that has a cross-sectional area that is less than the cross-sectional area of the elongated body 502. In some aspects, the distal end of the second folding section 514b includes a recess 517b such that the first folding section 514a can rotate with respect to the second folding section 514b. In some aspects, the proximal end of the second folding section 514b includes a recess 517b such that the second folding section 514b can rotate with respect to the third folding section 514c.

Continuing with FIGS. 5A-5C, the disclosure now turns to the second folding section 514b. The third folding section 514c is rotatably coupled to the elongated section 512 along a third folding axis FA″. In this manner, the third folding section 514c can rotate about the third folding axis FA′″ with respect to the elongated section 512. In some aspects, the third folding axis FA′″ is generally perpendicular to the longitudinal axis LAP of the periscope device 500 (e.g., the longitudinal axis of the folding section 514). For example, the third folding axis FA′″ can be generally perpendicular to the longitudinal axis of the third folding section 514c and/or generally perpendicular to the longitudinal axis of the elongated section 512 (e.g., the longitudinal axis of the distal end of the elongated section 512). In some aspects, the third folding axis FA′″ is generally parallel to the first folding axis FA′. In some aspects, the third folding axis FA′″ is generally parallel to the second folding axis FA″. As previously discussed, the third folding section 514c can be constructed of a rigid or semi-rigid material, such that the longitudinal axis of the third folding section 514c remains generally linear even when third folding section 514c is rotated about the third folding axis FA′″.

In some aspects, the third folding axis FA′″ is defined by a third pin 510c. In other words, the third pin 510c secures the third folding section 514c to the elongated section 512, while allowing the third folding section 514c to rotate about the third folding axis FA′″. In some aspects, as best illustrated in FIG. 5C, the proximal end of the third folding section 514c can include one or more knuckles 511c and the distal end of the elongated section 512 can include one or more knuckles 511d. The third pin 510c can join the knuckles 511c, 511d (e.g., the knuckles 511c, 511d can receive the third pin 510c therethrough) to rotatably couple the third folding section 514c to the elongated section 512.

A third angle of rotation can be defined as the angle between the longitudinal axis of the third folding section 514c and the longitudinal axis of the elongated section 512. In other words, the third angle of rotation changes as the third folding section 514c rotates about the third folding axis FA′″ with respect to the elongated section 512. In some aspects, the third angle of rotation can range between 180-degrees (i.e., the longitudinal axis of the third folding section 514c is generally parallel to or colinear with the longitudinal axis of the elongated section 512) to approximately 90-degrees (i.e., the longitudinal axis of the third folding section 514c is generally perpendicular to the longitudinal axis of the elongated section 512). In some aspects, when the periscope device 500 is in an undeployed position, the third angle of rotation is generally 180-degrees. In some aspects, when the periscope device 500 is in a deployed position, the third angle of rotation is between 60-degrees and 120-degrees. In some aspects, when the periscope device 500 is in a deployed position, the third angle of rotation is generally 90-degrees.

In some aspects, the third folding axis FA′″ is generally perpendicular to both the longitudinal axis of the third folding section 514c and the longitudinal axis of the elongated section 512, regardless of the third angle of rotation. For example, when the third angle of rotation is generally 180-degrees (e.g., when the periscope device 500 is undeployed), the third folding axis FA′″ is generally perpendicular to both the longitudinal axis of the third folding section 514c and the longitudinal axis of the elongated section 512. When the third angle of rotation is generally 90-degrees (e.g., when the periscope device 500 is deployed), the third folding axis FA′″ is generally perpendicular to both the longitudinal axis of the third folding section 514c and the longitudinal axis of the elongated body. In some aspects, the longitudinal axis of the third folding section 514c is generally coplanar with the longitudinal axis of the elongated section 512, regardless of the third angle of rotation. For example, when the third angle of rotation is generally 180-degrees, the longitudinal axis of the third folding section 514c is generally coplanar with the longitudinal axis of the elongated section 512 (e.g., the longitudinal axis at the distal end of the elongated section 512). When the third angle of rotation is generally 90-degrees, the longitudinal axis of the third folding section 514c is generally coplanar with the longitudinal axis of the elongated section 512.

The third folding section 514c can include one or more recesses 517 (e.g., 517c), as best illustrated in FIG. 5B and FIG. 5C. Each of the recesses 517c define a portion of the third folding section 514c that has a cross-sectional area that is less than the cross-sectional area of the elongated body 502. In some aspects, the distal end of the third folding section 514c includes a recess 517c, as illustrated in FIG. 5C, such that the second folding section 514b can rotate with respect to the third folding section 514c. In some aspects, the proximal end of the third folding section 514c includes a recess 517c, as illustrated in FIG. 5B, such that the third folding section 514c can rotate with respect to the elongated section 512. In some aspects, the distal end of the elongated section 512 includes a recess 517d such that the third folding section 514c can rotate with respect to the elongated section 512.

Turning now to FIGS. 6A-6D, one embodiment of a periscope device 500 is illustrated being used in conjunction with a duodenoscope 10. The periscope device 500 can be advanced through the instrument channel 22 of a duodenoscope 10 and deployed in the same or similar manner to the periscope device 100 (as illustrated in FIGS. 1A-3C) as previously described.

FIG. 6A illustrates a perspective view of a duodenoscope 10 alone (e.g., without a periscope device 500 therein). The distal end 506 of the periscope device 500 can be advanced through the instrument channel 22 of the duodenoscope 10, as illustrated for example in FIG. 6B, such that the distal end 506 of the periscope device 500 extends from the distal end of the instrument channel 22. Then periscope device 500 can be deployed, as illustrated for example in FIGS. 6C-6D to position the mirror 516 to reflect a view from the optical lens 26 of the duodenoscope 10 (e.g., reflect the side view to provide a forward-facing view). The first folding section 514a can be rotated about the first folding axis FA′ with respect to the second folding section 514b, as illustrated for example in FIG. 6C. The second folding section 514b can be rotated about the second folding axis FA″ (e.g., defined by the longitudinal axis of the second pin 510b, which is into the page) with respect to the third folding section 514c, as illustrated for example in FIG. 6D.

In some embodiments, as illustrated for example in FIGS. 6A-6D, the periscope device 500 can be deployed to position the mirror over the optical lens 26 of the duodenoscope 10 without requiring an elevator (e.g., actuating an elevator to raise a portion of the elongated body 502 of the periscope device 500). For example, the folding section 514 (or a portion of the folding section 514) can be elevated without an elevator 20. Rotation about one or more folding axis FA—for example first folding axis FA′, second folding axis FA″, and/or third folding axis FA′″—can properly position the periscope device 500 without the use of an elevator 20 in the duodenoscope 10. In one aspect, rotation of the second folding section 514b about the second folding axis FA″ elevates a portion of the folding section 514 without using an elevator in the duodenoscope 10.

FIG. 6D illustrates the periscope device 500 in a deployed position with the mirror 516 of the elongated body 502 positioned to reflect the view from the optical lens 26 of the duodenoscope 10, such that a forward-facing view is provided. In some aspects, the longitudinal axis LAP of the elongated body 502 (e.g., the longitudinal axis of the first folding section 514a) is perpendicular to a longitudinal axis of the optical lens LAO when the periscope device 500 is deployed, as illustrated for example in FIG. 6D. In some aspects, the longitudinal axis LAP of the elongated body 502 (e.g., the longitudinal axis of the first folding section 514a) is coplanar with the longitudinal axis of the optical lens LAO when the periscope device 500 is deployed.

FIG. 6D illustrates, for example. a light source 513 near the second folding axis FA″ (the second folding axis FA″ not visible in FIG. 6). In some aspects, during and/or after deployment of the periscope device 500, the longitudinal axis of the light source 513 can be generally parallel to the longitudinal axis LAD of the duodenoscope 10 such that when the light source 513 is illuminated (e.g., turned on) it illuminates at least a portion of the field of view (e.g., forward-facing view provided by the mirror 516). In some aspects, the longitudinal axis of the light source 513 can be generally perpendicular to the second folding axis FA″. For example, during and/or after deployment of the periscope device 500, the second folding axis FA″ can be generally perpendicular to the longitudinal axis LAD of the duodenoscope 10 and the longitudinal axis of the light source 513 can be generally parallel to the longitudinal axis LAD of the duodenoscope 10).

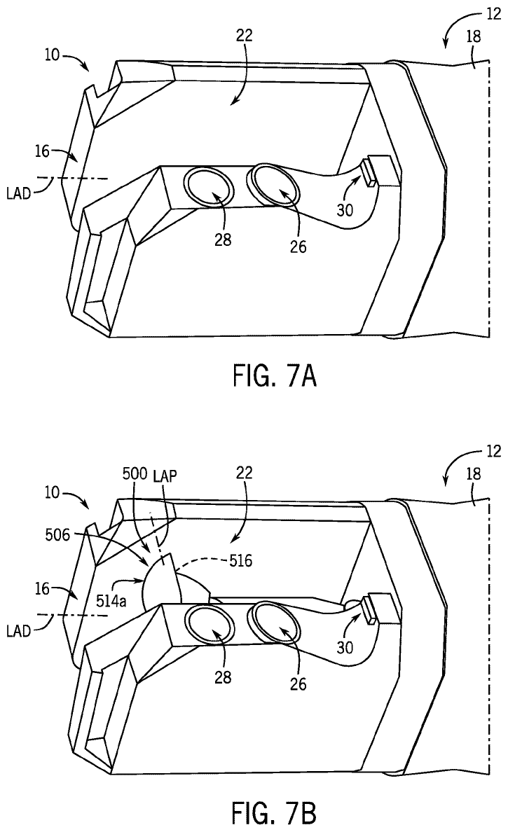

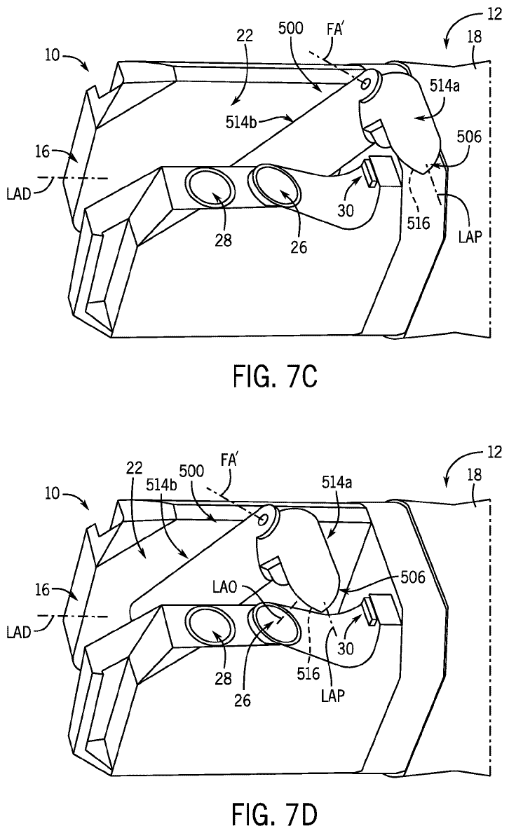

Turning now to FIGS. 7A-7D, one embodiment of a periscope device 500 is illustrated being used in conjunction with a duodenoscope 10. The periscope device 500 can be advanced through the instrument channel 22 of a duodenoscope 10 and deployed in the same or similar manner to the periscope device 100 (as illustrated in FIGS. 1A-3C) as previously described.

FIG. 7A illustrates a perspective view of a duodenoscope 10 alone (e.g., without a periscope device 500 therein). The distal end 506 of the periscope device 500 can be advanced through the instrument channel 22 of the duodenoscope 10, as illustrated for example in FIG. 7B, such that the distal end 506 of the periscope device 500 extends from the distal end of the instrument channel 22. Then periscope device 500 can be deployed, as illustrated for example in FIGS. 7B-7D to position the mirror 516 to reflect a view from the optical lens 26 of the duodenoscope 10 (e.g., reflect the side view to provide a forward-facing view). The first folding section 514a can be rotated about the first folding axis FA′ with respect to the second folding section 514b, as illustrated for example in FIGS. 7B-7C. The second folding section 514b can be rotated about the second folding axis FA″ with respect to the third folding section 514c, as illustrated for example in FIG. 7C. The elongated body 502 within the channel 22 of the duodenoscope 10 can be advanced or retracted to position the mirror 516 with respect to the optical lens 26 of the duodenoscope 10. For example. the elongated body 502 is illustrated as being advanced from FIG. 7C to FIG. 7D to position the mirror 516 in line with the optical lens 26, In some embodiments, as illustrated for example in FIGS. 7A-7D, the periscope device 500 can be deployed to position the mirror over the optical lens 26 of the duodenoscope 10 without requiring an elevator (e.g., actuating an elevator to raise a portion of the elongated body 502 of the periscope device 500).

FIG. 7D illustrates the periscope device 500 in a deployed position with the mirror 516 of the elongated body 502 positioned to reflect the view from the optical lens 26 of the duodenoscope 10, such that a forward-facing view is provided. In some aspects, the longitudinal axis LAP of the elongated body 502 (e.g., the longitudinal axis of the first folding section 514a) is perpendicular to a longitudinal axis LAO of the optical lens 26 when the periscope device 500 is deployed, as illustrated for example in FIG. 7D. In some aspects, the longitudinal axis LAP of the elongated body 502 (e.g., the longitudinal axis of the first folding section 514a) is coplanar with the longitudinal axis LAO of the optical lens 26 when the periscope device 500 is deployed. In some aspects, the longitudinal axis LAO of the optical lens 26 is generally perpendicular to the longitudinal axis LAD of the duodenoscope 10, as illustrated for example in FIGS. 2A-2C and FIGS. 4A-4C. However, in other examples, such as illustrated in FIGS. 7A-7D, the longitudinal axis LAO of the optical lens 26 defines an angle with respect to a line that is perpendicular to the longitudinal axis LAD of the duodenoscope 10. In some examples, the angle can be between 0-degrees and 15-degrees.

What is claimed is:

1. A periscope device for use with a duodenoscope, the periscope device comprising:
an elongated body defining a longitudinal axis and having a proximal end, a distal end, and an outer surface, the elongated body configured to be inserted into a working lumen of the duodenoscope;
a folding section of the elongated body, the folding section configured to rotate about a folding axis;
a mirror coupled to the folding section of the elongated body; and
a deployment mechanism coupled to the proximal end of the elongated body,
wherein deploying the deployment mechanism causes the folding section to rotate about the folding axis.

2. The periscope device of claim 1, wherein the folding axis defines an elongated section of the elongated body, the elongated section extending from the proximal end of the elongated body to the folding axis, the folding section extending from the distal end of the elongated body to the folding axis.

3. The periscope device of claim 2, wherein the elongated section is constructed of a flexible material and the folding section is constructed of a rigid or semi-rigid material.

4. The periscope device of claim 2, wherein the folding axis is defined by a pin such that the folding section rotates about the pin, wherein the folding section includes one or more knuckles and the elongated section includes one or more knuckles, the one or more knuckles of the folding section corresponding to the one or more knuckles of the elongated section, the one or more knuckles of the folding section and the one or more knuckles of the elongated section having the pin therethrough.

5. The periscope device of claim 2, wherein the deployment mechanism is configured to cause the folding section to rotate about the folding axis to an angle of rotation with respect elongated section, wherein the angle of rotation is between 0-degrees and 180-degrees.

6. The periscope device of claim 2, wherein the folding axis defines a fold-axis angle with respect to the longitudinal axis of the elongated body, wherein the fold-axis angle is approximately 45-degrees.

7. The periscope device of claim 1, wherein the folding section is a first folding section and the folding axis is a first folding axis, the periscope device further comprising a second folding section configured to rotate about a second folding axis, wherein deploying the deployment mechanism causes the second folding section to rotate about the second folding axis.

8. The periscope device of claim 7, further comprising a third folding section configured to rotate about a third folding axis, wherein deploying the deployment mechanism causes the third folding section to rotate about the third folding axis.

9. The periscope device of claim 8, wherein the first folding axis, the second folding axis, and the third folding axis are each generally perpendicular to the longitudinal axis of the elongated body.

10. The periscope device of claim 8, wherein a longitudinal axis of the first folding section is generally coplanar with a longitudinal axis of the second folding section, wherein the longitudinal axis of the second folding section is generally coplanar with a longitudinal axis of the third folding section.

11. The periscope device of claim 8, wherein the longitudinal axis of the first folding section is perpendicular to a longitudinal axis of an optical lens of the duodenoscope.

12. (canceled)

13. (canceled)

14. The periscope device of claim 1, wherein the deployment mechanism causes the elongated body to transition from an undeployed position to a deployed position, wherein the periscope device does not obstruct a side-facing view from an optical lens of the duodenoscope when the periscope device is in the undeployed position, wherein the folding section reflects the side-facing view from the optical lens to provide a forward-facing view when the periscope device is in the deployed position.

15. The periscope device of claim 14, wherein a longitudinal axis of the forward-facing view is generally perpendicular to a longitudinal axis of the optical lens.

16. The periscope device of claim 2, wherein the elongated body has a generally circular cross section, wherein the folding section and the elongated section each include a recess to accommodate rotation of the folding section about the folding axis.

17. The periscope device of claim 1, further comprising a light source coupled to the folding section, the light source defining a longitudinal axis, wherein the longitudinal axis of the light source is generally parallel to a longitudinal axis of the duodenoscope after the folding section rotates about the folding axis.

18. (canceled)

19. The periscope device of claim 1, wherein the folding axis of the elongated body is oriented at approximately a 45-degree angle with respect to the longitudinal axis of the elongated body.

20-25. (canceled)

26. A system for providing forward viewing with a duodenoscope, the system comprising:
the duodenoscope comprising:
a duodenoscope body defining a longitudinal axis and having a proximal end, a distal end, and an outer surface;
an optical lens located on the outer surface near the distal end of the duodenoscope body and configured to provide imaging;
a working lumen within the duodenoscope body and defining a proximal end and a distal end, wherein the distal end of the working lumen is near the optical lens; and
an elevator associated with the distal end of the working lumen, the elevator operable to adjust to an angle of elevation; and
a periscope device comprising:
an elongated body defining a longitudinal axis and having a proximal end, a distal end, and an outer surface;
a folding axis of the elongated body defining an elongated section of the elongated body and a folding section of the elongated body;
a mirror attached to the folding section of the elongated body; and
a deployment mechanism connected to the proximal end of the elongated body,
wherein the deployment mechanism is configured to rotate the folding section of the elongated body about the folding axis at an angle of rotation with respect to the elongated section of the elongated body.

27. The system of claim 26, wherein the folding axis of the elongated body is oriented at approximately a 45-degree angle with respect to the longitudinal axis of the elongated body.

28-33. (canceled)

34. A method of using a periscope device to provide forward viewing with a duodenoscope, the method comprising:
inserting a distal end of an elongated body of the periscope device into a proximal end of a working lumen of a duodenoscope, wherein a folding axis is incorporated into the elongated body, the folding axis defining an elongated section of the elongated body and a folding section of the elongated body, wherein a mirror is attached to the folding section of the elongated body;
advancing the elongated body of the periscope device until the folding section of the elongated body extends from a distal end of the working lumen of the duodenoscope;
actuating a deployment mechanism connected to the proximal end of the elongated body of the periscope device, thereby rotating the folding section of the elongated body about the folding axis at an angle of rotation with respect to the elongated section of the elongated body; and
orienting the mirror to reflect a view from an optical lens of the duodenoscope at a viewing angle.

35. The method of claim 34 wherein the angle of rotation is between 0-degrees and 180-degrees.

36. The method of claim 34, wherein the viewing angle is approximately 45-degrees.

37.-41. (canceled)
